(19) United States
(12) Patent Application Publication
(10) Pub. No.: US 2023/0218883 A1
(43) Pub. Date: Jul. 13, 2023

(54) PINCH VALVE FOR FLUID INJECTOR SYSTEM

(71) Applicant: BAYER HEALTHCARE LLC, WHIPPANY, NJ (US)

(72) Inventors: ANDREW NAPLES, MARS, PA (US); MICHAEL SPOHN, FENELTON, PA (US); KEVIN COWAN, ALLISON PARK, PA (US); CHRISTOPHER CAPONE, PITTSBURGH, PA (US); EDDEN RABIN, WEXFORD, PA (US); JAROSLAW WLODARCZYK, LOWER BURRELL, PA (US)

(21) Appl. No.: 17/997,032

(22) PCT Filed: Apr. 29, 2021
(86) PCT No.: PCT/US2021/029963

§ 371 Date: Oct. 25, 2022

Related U.S. Application Data
(60) Provisional application No. 63/019,013, filed on May 1, 2020.

(51) Int. Cl. A61M 39/28; A61M 5/145; A61M 5/168

(52) U.S. Cl. CPC A61M 39/285; A61M 5/1452; A61M 5/16827

(57) ABSTRACT

A fluid injector system includes at least one injector for pressurizing and delivering at least one fluid from at least one fluid reservoir, a first flexible tube having a first lumen, a second flexible tube having a second lumen, and a valve assembly configured to selectively and reversibly compress the first flexible tube and the second flexible tube to open and close the first lumen and the second lumen. The valve assembly includes a first anvil moveable between a retracted position in which the first lumen is at least partially pen and an extended position in which the first anvil closes the first lumen, a second anvil moveable between a retracted position in which the second lumen is at least partially open and an extended position in which the second anvil closes the second lumen, and at least one eccentric cam rotatable to move the first anvil and the second anvil between the retracted and extended positions.

### CROSS-REFERENCE TO RELATED APPLICATIONS

The present application claims the benefit of U.S. Provisional Patent Application No. 63/019,013, filed on May 1, 2020, the disclosure of which is hereby incorporated by reference in its entirety.

### BACKGROUND OF THE DISCLOSURE

### Field of the Disclosure

The present disclosure is related to the field of valves for a medical injector system. More particularly, the present disclosure is directed to valves for selectively opening and closing flexible tubing of a fluid path set.

### Description of Related Art

In many medical diagnostic and therapeutic procedures, a patient is injected with one or more fluids. In recent years, a number of injector-actuated syringes and powered injectors for pressurized injection of fluids have been developed for use in procedures such as angiography (CV), computed tomography (CT), molecular imaging (such as PET imaging), and magnetic resonance imaging (MRI). In these procedures, a medical fluid, such as a contrast agent, may be used to highlight certain vasculature systems, internal organs, or portions of the body during an imaging process. The medical fluid may be delivered to the patient by the powered injector by one or more pump, syringe, or combination thereof.

When preparing to inject a medical fluid into a patient, it is important that the injection reservoir is fully filled with the medical fluid and air removed to avoid inadvertent injection of air into the patient. In certain procedures such as angiography, even small quantities of air may present a concern if injected into the vasculature during the injection procedure. The inclusion of air detectors, either at the syringe or on the fluid path may help notify the user that air is present and there is a possibility of the air being injected with the contrast. When air is detected, stopping the injection procedure prior to the air reaching the patient's vasculature is desired. However, due to system pressurization resulting in compliance, i.e., swelling or deflection of system components particularly at pressures used for injection of medical fluids during certain procedures, simply stopping the motor of the powered injector may not immediately stop the flow of fluid through the fluid path set and into the patient.

Further, various components of the injector systems need to be isolated during certain stages of an injection procedure such as filling, purging, and injection. For example, the administration line connected to the patient should be isolated from the fluid reservoir when the fluid reservoir is being filled from a bulk fluid source. Similarly, the bulk fluid source should be isolated from the fluid reservoir during injection of the medical fluid.

### SUMMARY OF THE DISCLOSURE

In view of the foregoing, there exists a need for devices and systems for rapidly stopping the flow of fluid in tubing, and for isolating components of a fluid injector system. Accordingly, embodiments of the present disclosure are directed to a fluid injector system including at least one injector for pressurizing and delivering at least one fluid from at least one fluid reservoir, a first flexible tube having a first lumen, the first flexible tube in fluid communication with the at least one fluid reservoir and configured for fluid communication with a bulk fluid source, a second flexible tube having a second lumen, the second flexible tube in fluid communication with the at least one fluid reservoir and configured for fluid communication with a patient administration line, and a valve assembly configured to selectively and reversibly compress the first flexible tube and the second flexible tube to open and close the first lumen and the second lumen. The valve assembly includes a first anvil moveable between a retracted position in which the first lumen is at least partially open and an extended position in which the first anvil reversibly compresses the first flexible tube to close the first lumen, a second anvil moveable between a retracted position in which the second lumen is at least partially open and an extended position in which the second anvil reversibly compresses the second flexible tube to close the second lumen, and at least one eccentric cam rotatable to move the first anvil and the second anvil between the retracted position and the extended position.

In some embodiments, valve assembly is movable between a first position in which the first lumen and the second lumen are at least partially open, a second position in which the valve assembly closes the first lumen and the second lumen is at least partially open, a third position in which the first lumen is at least partially open and the valve assembly closes the second lumen, and a fourth position in which the valve assembly closes the first lumen and the second lumen.

In some embodiments, the first anvil is moveable along a first axis and the second anvil is moveable along a second axis. In some embodiments, the first axis is oriented at approximately 90° relative to the second axis.

In some embodiments, the valve assembly further includes a first biasing element biasing the first anvil toward the extended position, and a second biasing element biasing the second anvil toward the extended position.

In some embodiments, the valve assembly further includes a first backing plate biased toward the first flexible tube to provide a pressure against the first flexible tube, and a second backing plate biased toward the second flexible tube to provide a pressure against the second flexible tube. In some embodiments, the pressures provided by the first backing plate and the second backing plate are adjustable.

In some embodiments, the at least one eccentric cam includes a single cam engaging both the first anvil and the second anvil. In some embodiments, the at least one eccentric cam includes a first cam engaging the first anvil and a second cam engaging the second anvil. The first cam and the second cam share a rotation axis.

In some embodiments, the at least one eccentric cam includes at least one constant radius section. With the first anvil engaging the constant radius section, rotation of the at least one eccentric cam over a span of the constant radius section does not move the first anvil between the retracted position and the extended position. With the second anvil engaging the constant radius section, rotation of the at least one eccentric cam over a span of the constant radius section does not move the second anvil between the retracted position and the extended position.

In some embodiments, the first flexible tube is connected to and in fluid communication with the second flexible tube between the at least one fluid reservoir and the valve assembly.

In some embodiments, the valve assembly further includes a backing plate having a groove for receiving the first flexible tube, and the first anvil includes a projection for reversibly compressing the first flexible tube against the groove of the backing plate.

In some embodiments, the fluid injector system further including at least one air detector associated with at least one of the first flexible tube and the second flexible tube, and a controller programmed or configured to move the valve assembly to the fourth position in response to detection of at least one air bubble by the at least one air detector.

Other embodiments of the present disclosure are directed to a fluid injector system including, at least one injector for pressurizing and delivering at least one fluid from at least one fluid reservoir, a first flexible tube having a first lumen, the first flexible tube in fluid communication with the at least one fluid reservoir and configured for fluid communication with a bulk fluid source, and a valve assembly configured to selectively and reversibly compress the first flexible tube to open and close the first lumen. The valve assembly includes a first backing plate configured for receiving the first flexible tube, a first anvil moveable between a retracted position and an extended position, wherein, in the extended position, the first anvil is configured to reversibly compress the first flexible tube against the first backing plate, and at least one driving element configured to move the first anvil between the retracted position and the extended position. The first backing plate is biased toward the first flexible tube to provide a pressure against the first flexible tube.

In some embodiments, the fluid injector system further includes a second flexible tube having a second lumen, the second flexible tube in fluid communication with the at least one fluid reservoir and configured for fluid communication with a patient administration line. The valve assembly is configured to selectively and reversibly compress the second flexible tube to open and close second lumen. The valve assembly further includes a second backing plate configured for receiving the second flexible tube, and a second anvil moveable between a retracted position and an extended position. In the extended position, the second anvil is configured to reversibly compress the second flexible tube against the second backing plate. The at least one driving element is configured to move the second anvil between the retracted position and the extended position. The second backing plate is biased toward the second flexible tube to provide a pressure against the second flexible tube.

In some embodiments, the at least one driving element includes at least one eccentric cam. The at least one eccentric cam is rotatable between a first position in which the first anvil and the second anvil are in retracted position, a second position in which the first anvil is in the extended position and the second anvil is in the retracted position, a third position in which the first anvil is in the retracted position and the second anvil is in the extended position, and a fourth position in which the first anvil and the second anvil are in the extended position.

In some embodiments, the at least one eccentric cam includes a single cam engaging both the first anvil and the second anvil. In some embodiments, the at least one eccentric cam includes a first cam engaging the first anvil and a second cam engaging the second anvil. The first cam and the second cam share a rotation axis.

In some embodiments, the at least one eccentric cam has a constant radius section. With the first anvil engaging the constant radius section, rotation of the at least one eccentric cam does not move the first anvil between the retracted position and the extended position.

In some embodiments, the at least one driving element includes at least one parallelogram linkage including at least one pair of legs rotatably connected to the first anvil. The at least one parallelogram linkage is moveable between a first position in which the first anvil and the second anvil are in retracted position, a second position in which the first anvil is in the extended position and the second anvil is in the retracted position, a third position in which the first anvil is in the retracted position and the second anvil is in the extended position, and a fourth position in which the first anvil and the second anvil are in the extended position.

In some embodiments, the first anvil is moveable along a first axis and the second anvil is moveable along a second axis. In some embodiments, the first axis is oriented at approximately 90° relative to the second axis.

In some embodiments, the valve assembly further includes a first biasing element biasing the first anvil toward the extended position.

In some embodiments, the first backing plate includes a groove configured for receiving the first flexible tube. The first anvil includes a projection configured for reversibly compressing the first flexible tube against the groove of the first backing plate.

Other embodiments of the present disclosure are directed to a valve for a fluid injector system. The valve includes a first backing plate configured for receiving the first flexible tube, a first anvil moveable between a retracted position and an extended position, wherein, in the extended position, the first anvil is configured to reversibly compress a first flexible tube against the first backing plate, and at least one driving element configured to move the first anvil between the retracted position and the extended position. The first backing plate is biased toward the first flexible tube to provide a pressure against the first flexible tube.

In some embodiments, the valve further includes a second backing plate configured for receiving a second flexible tube and a second anvil moveable between a retracted position and an extended position. In the extended position, the second anvil is configured to reversibly compress the second flexible tube against the second backing plate. The at least one driving element is configured to move the second anvil between the retracted position and the extended position. The second backing plate is biased toward the second flexible tube to provide a pressure against the second flexible tube.

In some embodiments, the at least one driving element includes at least one eccentric cam. The at least one eccentric cam is rotatable between a first position in which the first anvil and the second anvil are in retracted position, a second position in which the first anvil is in the extended position and the second anvil is in the retracted position, a third position in which the first anvil is in the retracted position and the second anvil is in the extended position, and a fourth position in which the first anvil and the second anvil are in the extended position.

In some embodiments, the at least one eccentric cam includes a single cam engaging both the first anvil and the second anvil. In some embodiments, the at least one eccentric cam includes a first cam engaging the first anvil and a second cam engaging the second anvil. The first cam and the second cam share a rotation axis.

In some embodiments, the at least one eccentric cam has a constant radius section. With the first anvil engaging the constant radius section, rotation of the at least one eccentric cam does not move the first anvil between the retracted position and the extended position.

In some embodiments, the at least one driving element includes at least one parallelogram linkage including at least one pair of legs rotatably connected to the first anvil. The at least one parallelogram linkage is moveable between a first position in which the first anvil and the second anvil are in retracted position, a second position in which the first anvil is in the extended position and the second anvil is in the retracted position, a third position in which the first anvil is in the retracted position and the second anvil is in the extended position, and a fourth position in which the first anvil and the second anvil are in the extended position.

In some embodiments, the first anvil is moveable along a first axis and the second anvil is moveable along a second axis. In some embodiments, the first axis is oriented at approximately 90° relative to the second axis.

In some embodiments, the valve further includes a first biasing element biasing the first anvil toward the extended position.

In some embodiments, the first backing plate includes a groove configured for receiving the first flexible tube. The first anvil includes a projection configured for reversibly compressing the first flexible tube against the groove of the first backing plate.

Other embodiments of the present disclosure are directed to a valve for a fluid injector system. The valve includes a backing plate configured for receiving a flexible tube, and an anvil moveable between a retracted position and an extended position. In the extended position, the anvil is configured to reversibly compress the flexible tube against the backing plate. The valve further includes a primary cam configured to engage the anvil to move the anvil from the retracted position and the extended position, and a secondary cam configured to engage a boss of the anvil to move the anvil from the extended position to the retracted position.

In some embodiments, a radius of the primary cam increases in a first direction of rotation, and a radius of the secondary cam increases in a second directing of rotation opposite the first direction of rotation. In some embodiments, the primary cam and the secondary cam are configured to rotate in unison. In some embodiments, a portion of the primary cam having a maximum radius engages the anvil when the anvil is in the extended position. In some embodiments, a portion of the secondary cam having a minimum radius engages the boss when the anvil is in the extended position.

In some embodiments, a portion of the primary cam having a minimum radius engages the anvil when the anvil is in the retracted position. In some embodiments, a portion of the secondary cam having a maximum radius engages the boss when the anvil is in the retracted position.

In some embodiments, the anvil includes at least one finger configured to engage the flexible tube in the retracted position to retain the flexible tube against the backing plate.

In some embodiments, the valve further includes a tubing detector configured to detect the presence or absence of the flexible tube on the backing plate. In some embodiments, the tubing detector includes at least one of a proximity sensor, an optical sensor, a pressure sensor, a pressure plate, or a limit switch.

Further embodiments of the present disclosure are described in the following numbered clauses:

Clause 1. A fluid injector system, comprising: at least one injector for pressurizing and delivering at least one fluid from at least one fluid reservoir; a first flexible tube having a first lumen, the first flexible tube in fluid communication with the at least one fluid reservoir and configured for fluid communication with a bulk fluid source; a second flexible tube having a second lumen, the second flexible tube in fluid communication with the at least one fluid reservoir and configured for fluid communication with a patient administration line; and a valve assembly configured to selectively and reversibly compress the first flexible tube and the second flexible tube to open and close the first lumen and the second lumen, the valve assembly comprising: a first anvil moveable between a retracted position in which the first lumen is at least partially open and an extended position in which the first anvil reversibly compresses the first flexible tube to close the first lumen; a second anvil moveable between a retracted position in which the second lumen is at least partially open and an extended position in which the second anvil reversibly compresses the second flexible tube to close the second lumen; and at least one eccentric cam rotatable to move the first anvil and the second anvil between the retracted position and the extended position.

Clause 2. The fluid injector system of clause 1, wherein the valve assembly is movable between: a first position in which the first lumen and the second lumen are at least partially open; a second position in which the valve assembly closes the first lumen and the second lumen is at least partially open; a third position in which the first lumen is at least partially open and the valve assembly closes the second lumen; and a fourth position in which the valve assembly closes the first lumen and the second lumen.

Clause 3. The fluid injector system of clause 1 or 2, wherein the first anvil is moveable along a first axis, and wherein the second anvil is moveable along a second axis.

Clause 4. The fluid injector system of any of clauses 1 to 3, wherein the first axis is oriented at approximately 90° relative to the second axis.

Clause 5. The fluid injector system of any of clauses 1 to 4, wherein the valve assembly further comprises: a first biasing element biasing the first anvil toward the extended position; and a second biasing element biasing the second anvil toward the extended position.

Clause 6. The fluid injector system of any of clauses 1 to 5, wherein the valve assembly further comprises: a first backing plate biased toward the first flexible tube to provide a pressure against the first flexible tube; and a second backing plate biased toward the second flexible tube to provide a pressure against the second flexible tube.

Clause 7. The fluid injector system of any of clauses 1 to 6, where the pressures provided by the first backing plate and the second backing plate are adjustable.

Clause 8. The fluid injector system of any of clauses 1 to 7, wherein the at least one eccentric cam comprises a single cam engaging both the first anvil and the second anvil.

Clause 9. The fluid injector system of any of clauses 1 to 8, wherein the at least one eccentric cam comprises: a first cam engaging the first anvil; and a second cam engaging the second anvil, wherein the first cam and the second cam share a rotation axis.

Clause 10. The fluid injector system of any of clauses 1 to 9, wherein the at least one eccentric cam comprises at least one constant radius section, wherein, with the first anvil engaging the constant radius section, rotation of the at least one eccentric cam over a span of the constant radius section does not move the first anvil between the retracted position and the extended position, and wherein, with the second anvil engaging the constant radius section, rotation of the at least one eccentric cam over a span of the constant radius section does not move the second anvil between the retracted position and the extended position.

Clause 11. The fluid injector system of any of clauses 1 to 10, wherein the first flexible tube is connected to and in fluid communication with the second flexible tube between the at least one fluid reservoir and the valve assembly.

Clause 12. The fluid injector system of any of clauses 1 to 11, wherein the valve assembly further comprises: a backing plate having a groove for receiving the first flexible tube, and wherein the first anvil comprises a projection for reversibly compressing the first flexible tube against the groove of the backing plate.

Clause 13. The fluid injector system of any of clauses 1 to 12, further comprising at least one air detector associated with at least one of the first flexible tube and the second flexible tube; and a controller programmed or configured to move the valve assembly to the fourth position in response to detection of at least one air bubble by the at least one air detector.

Clause 14. A fluid injector system, comprising: at least one injector for pressurizing and delivering at least one fluid from at least one fluid reservoir; a first flexible tube having a first lumen, the first flexible tube in fluid communication with the at least one fluid reservoir and configured for fluid communication with a bulk fluid source; a valve assembly configured to selectively and reversibly compress the first flexible tube to open and close the first lumen, the valve assembly comprising: a first backing plate configured for receiving the first flexible tube; a first anvil moveable between a retracted position and an extended position, wherein, in the extended position, the first anvil is configured to reversibly compress the first flexible tube against the first backing plate; and at least one driving element configured to move the first anvil between the retracted position and the extended position, wherein the first backing plate is biased toward the first flexible tube to provide a pressure against the first flexible tube.

Clause 15. The fluid injector system of clause 14, further comprising: a second flexible tube having a second lumen, the second flexible tube in fluid communication with the at least one fluid reservoir and configured for fluid communication with a patient administration line, wherein the valve assembly is configured to selectively and reversibly compress the second flexible tube to open and close second lumen, the valve assembly further comprising: a second backing plate configured for receiving the second flexible tube; and a second anvil moveable between a retracted position and an extended position, wherein, in the extended position, the second anvil is configured to reversibly compress the second flexible tube against the second backing plate, wherein the at least one driving element is configured to move the second anvil between the retracted position and the extended position, and wherein the second backing plate is biased toward the second flexible tube to provide a pressure against the second flexible tube.

Clause 16. The fluid injector system of clause 14 or 15, wherein the at least one driving element comprises at least one eccentric cam, and wherein the at least one eccentric cam is rotatable between: a first position in which the first anvil and the second anvil are in retracted position; a second position in which the first anvil is in the extended position and the second anvil is in the retracted position; a third position in which the first anvil is in the retracted position and the second anvil is in the extended position; and a fourth position in which the first anvil and the second anvil are in the extended position.

Clause 17. The fluid injector system of any of clauses 14 to 16, wherein the at least one eccentric cam comprises a single cam engaging both the first anvil and the second anvil.

Clause 18. The fluid injector system of any of clauses 14 to 17, wherein the at least one eccentric cam comprises: a first cam engaging the first anvil; and a second cam engaging the second anvil, wherein the first cam and the second cam share a rotation axis.

Clause 19. The fluid injector system of any of clauses 14 to 18, wherein the at least one eccentric cam has a constant radius section, and wherein, with the first anvil engaging the constant radius section, rotation of the at least one eccentric cam does not move the first anvil between the retracted position and the extended position.

Clause 20. The fluid injector system of any of clauses 14 to 19, wherein the at least one driving element comprises at least one parallelogram linkage comprising at least one pair of legs rotatably connected to the first anvil, and wherein the at least one parallelogram linkage is moveable between: a first position in which the first anvil and the second anvil are in retracted position; a second position in which the first anvil is in the extended position and the second anvil is in the retracted position; a third position in which the first anvil is in the retracted position and the second anvil is in the extended position; and a fourth position in which the first anvil and the second anvil are in the extended position.

Clause 21. The fluid injector system of any of clauses 14 to 20, wherein the first anvil is moveable along a first axis, wherein the second anvil is moveable along a second axis.

Clause 22. The fluid injector system any of clauses 14 to 21, wherein the first axis is oriented at approximately 90° relative to the second axis.

Clause 23. The fluid injector system of any of clauses 14 to 22, wherein the valve assembly further comprises: a first biasing element biasing the first anvil toward the extended position.

Clause 24. The fluid injector system of any of clauses 14 to 23, wherein the first backing plate comprises a groove configured for receiving the first flexible tube, and wherein the first anvil comprises a projection configured for reversibly compressing the first flexible tube against the groove of the first backing plate.

Clause 25. A valve for a fluid injector system, the valve comprising: a first backing plate configured for receiving the first flexible tube; a first anvil moveable between a retracted position and an extended position, wherein, in the extended position, the first anvil is configured to reversibly compress a first flexible tube against the first backing plate; and at least one driving element configured to move the first anvil between the retracted position and the extended position, wherein the first backing plate is biased toward the first flexible tube to provide a pressure against the first flexible tube.

Clause 26. The valve of clause 25, further comprising: a second backing plate configured for receiving a second flexible tube; and a second anvil moveable between a retracted position and an extended position, wherein, in the extended position, the second anvil is configured to reversibly compress the second flexible tube against the second backing plate, wherein the at least one driving element is configured to move the second anvil between the retracted position and the extended position, and wherein the second backing plate is biased toward the second flexible tube to provide a pressure against the second flexible tube.

Clause 27. The valve of clause 25 or 26, wherein the at least one driving element comprises at least one eccentric cam, and wherein the at least one eccentric cam is rotatable between: a first position in which the first anvil and the second anvil are in retracted position; a second position in which the first anvil is in the extended position and the second anvil is in the retracted position; a third position in which the first anvil is in the retracted position and the second anvil is in the extended position; and a fourth position in which the first anvil and the second anvil are in the extended position.

Clause 28. The valve of any of clauses 25 to 27, wherein the at least one eccentric cam comprises a single cam engaging both the first anvil and the second anvil.

Clause 29. The valve of any of clauses 25 to 28, wherein the at least one eccentric cam comprises: a first cam engaging the first anvil; and a second cam engaging the second anvil, wherein the first cam and the second cam share a rotation axis.

Clause 30. The valve of any of clauses 25 to 29, wherein the at least one eccentric cam has a constant radius section, and wherein, with the first anvil engaging the constant radius section, rotation of the at least one eccentric cam does not move the first anvil between the retracted position and the extended position.

Clause 31. The valve of any of clauses 25 to 30, wherein the at least one driving element comprises at least one parallelogram linkage comprising at least one pair of legs rotatably connected to the first anvil, and wherein the at least one parallelogram linkage is moveable between: a first position in which the first anvil and the second anvil are in retracted position; a second position in which the first anvil is in the extended position and the second anvil is in the retracted position; a third position in which the first anvil is in the retracted position and the second anvil is in the extended position; and a fourth position in which the first anvil and the second anvil are in the extended position.

Clause 32. The valve of any of clauses 25 to 31, wherein the first anvil is moveable along a first axis, wherein the second anvil is moveable along a second axis.

Clause 33. The valve of any of clauses 25 to 32, wherein the first axis is oriented at approximately 90° relative to the second axis.

Clause 34. The valve of any of clauses 25 to 33, further comprising: a first biasing element biasing the first anvil toward the extended position.

Clause 35. The valve of any of clauses 25 to 34, wherein the first backing plate comprises a groove configured for receiving the first flexible tube, and wherein the first anvil comprises a projection configured for reversibly compressing the first flexible tube against the groove of the first backing plate.

Clause 36. A valve for a fluid injector system, the valve comprising: a backing plate configured for receiving a flexible tube; an anvil moveable between a retracted position and an extended position, wherein, in the extended position, the anvil is configured to reversibly compress the flexible tube against the backing plate; a primary cam configured to engage the anvil to move the anvil from the retracted position and the extended position; and a secondary cam configured to engage a boss of the anvil to move the anvil from the extended position to the retracted position.

Clause 37. The valve of clause 36, wherein a radius of the primary cam increases in a first direction of rotation, and wherein a radius of the secondary cam increases in a second directing of rotation opposite the first direction of rotation.

Clause 38. The valve of clause 36 or 37, wherein the primary cam and the secondary cam are configured to rotate in unison.

Clause 39. The valve of any of clauses 36-38, wherein a portion of the primary cam having a maximum radius engages the anvil when the anvil is in the extended position.

Clause 40. The valve of any of clauses 36-39, wherein a portion of the secondary cam having a minimum radius engages the boss when the anvil is in the extended position.

Clause 41. The valve of any of clauses 36-40, wherein a portion of the primary cam having a minimum radius engages the anvil when the anvil is in the retracted position.

Clause 42. The valve of any of clauses 36-41, wherein a portion of the secondary cam having a maximum radius engages the boss when the anvil is in the retracted position.

Clause 43. The valve of any of clauses 36-42, wherein the anvil comprises at least one finger configured to engage the flexible tube in the retracted position to retain the flexible tube against the backing plate.

Clause 44. The valve of any of clauses 36-43, further comprising a tubing detector configured to detect the presence or absence of the flexible tube on the backing plate.

Clause 45. The valve of any of clauses 36-44, wherein the tubing detector comprises at least one of a proximity sensor, an optical sensor, a pressure sensor, a pressure plate, or a limit switch.

Further details and advantages of the various examples described in detail herein will become clear upon reviewing the following detailed description of the various examples in conjunction with the accompanying drawing figures.

### BRIEF DESCRIPTION OF THE DRAWINGS

FIG. 1 is a perspective view of a fluid injector system according to an embodiment of the present disclosure;

FIG. 2 is a schematic diagram of the fluid injector system according to an embodiment of the present disclosure;

FIG. 3 is a schematic diagram of the fluid injector system according to an embodiment of the present disclosure;

FIG. 4A is a side view of a pinch valve according to an embodiment of the present disclosure, shown in an open position;

FIG. 4B is a side view of a pinch valve according to an embodiment of the present disclosure, shown in an open position;

FIG. 5A is a side view of the pinch valve of FIG. 4A in a closed position;

FIG. 5B is a side view of the pinch valve of FIG. 4B in a closed position;

FIG. 6 is a front view of a pinch valve according to an embodiment of the present disclosure, shown in an open position;

FIG. 7 is a front view of the pinch valve of FIG. 6 in a closed position;

FIG. 8 is an exploded view of a pinch valve according to an embodiment of the present disclosure;

FIG. 9 is a perspective view of a pinch valve according to an embodiment of the present disclosure;

FIG. 10 is a top view of the pinch valve of FIG. 9;

FIG. 11 is a side view of the pinch valve of FIG. 9;

FIG. 12 is a schematic diagram of the pinch valve of FIG. 9;

FIG. 13 is a top view of an embodiment of a cam profile of the pinch valve of FIG. 9;

FIG. 14 is a perspective view of a pinch valve assembly according to an embodiment of the present disclosure;

FIG. 15 is a top view of the pinch valve assembly of FIG. 14;

FIG. 16 is a side view of the pinch valve assembly of FIG. 14;

FIG. 17 is a schematic diagram of the pinch valve assembly of FIG. 14;

FIG. 18 is a top view of an embodiment of a cam profile of the pinch valve assembly of FIG. 14;

FIG. 19 is a perspective view of a pinch valve assembly according to an embodiment of the present disclosure;

FIG. 20 is a top view of the pinch valve assembly of FIG. 19, shown in a first position;

FIG. 21 is a top view of the pinch valve assembly of FIG. 19, shown in a second position;

FIG. 22 is a top view of the pinch valve assembly of FIG. 19, shown in a third position;

FIG. 23 is a top view of the pinch valve assembly of FIG. 19, shown in a fourth position;

FIG. 24 is a perspective view of a pinch valve assembly according to an embodiment of the present disclosure;

FIG. 25 is a top view of the pinch valve assembly of FIG. 24, shown in a first position;

FIG. 26 is a top view of the pinch valve assembly of FIG. 24, shown in a second position;

FIG. 27 is a top view of the pinch valve assembly of FIG. 24, shown in a third position;

FIG. 28 is a top view of the pinch valve assembly of FIG. 24, shown in a fourth position;

FIG. 29 is a side view of a pinch valve according to an embodiment of the present disclosure in an open position;

FIG. 30 is a side view of the pinch valve of FIG. 29 in a closed position;

FIG. 31 is a perspective view of a pinch valve according to an embodiment of the present disclosure;

FIG. 32 is a section view of the pinch valve of FIG. 31 along line A-A;

FIG. 33 is a section view of the pinch valve of FIG. 32 along line B-B;

FIG. 34 is a section view of the pinch valve of FIG. 32 along line C-C;

FIG. 35 is a section view of FIG. 33 taken along line D-D;

FIG. 36 is a perspective view of the pinch valve of FIG. 31 in a first rotational position;

FIG. 37 is a perspective view of the pinch valve of FIG. 31 in a second rotational position;

FIG. 38 is a perspective view of the pinch valve of FIG. 31 in a third rotational position;

FIG. 39 is a perspective view of the pinch valve of FIG. 31 in a fourth rotational position;

FIG. 40 is a perspective view of the pinch valve of FIG. 31 in a fifth rotational position;

FIG. 41 is a perspective view of the pinch valve of FIG. 31 in a sixth rotational position;

FIG. 42 is a perspective view of a pinch valve according to an embodiment of the present disclosure, in a closed position;

FIG. 43 is a side cross-sectional view of the pinch valve of FIG. 42 in the closed position, taken along line E-E of FIG. 42;

FIG. 44 is a perspective view of the pinch valve of FIG. 42 in an open position;

FIG. 45 is a side view of the pinch valve of FIG. 42 in the open position.

### DETAILED DESCRIPTION OF THE DISCLOSURE

For purposes of the description hereinafter, the terms “upper”, “lower”, “right”, “left”, “vertical”, “horizontal”, “top”, “bottom”, “lateral”, “longitudinal”, and derivatives thereof shall relate to the disclosure as it is oriented in the drawing figures. When used in relation to a syringe of a multi-patient disposable set, the term “proximal” refers to a portion of a syringe nearest a piston for delivering fluid from a syringe.

Spatial or directional terms, such as “left”, “right”, “inner”, “outer”, “above”, “below”, and the like, are not to be considered as limiting as the invention can assume various alternative orientations.

All numbers used in the specification and claims are to be understood as being modified in all instances by the term “about”. The terms “approximately”, “about”, and “substantially” mean a range of plus or minus ten percent of the stated value.

As used herein, the term “at least one of” is synonymous with “one or more of”. For example, the phrase “at least one of A, B, and C” means any one of A, B, and C, or any combination of any two or more of A, B, and C. For example, “at least one of A, B, and C” includes one or more of A alone; or one or more B alone; or one or more of C alone; or one or more of A and one or more of B; or one or more of A and one or more of C; or one or more of B and one or more of C; or one or more of all of A, B, and C. Similarly, as used herein, the term “at least two of” is synonymous with “two or more of”. For example, the phrase “at least two of D, E, and F” means any combination of any two or more of D, E, and F. For example, “at least two of D, E, and F” includes one or more of D and one or more of E; or one or more of D and one or more of F; or one or more of E and one or more of F; or one or more of all of D, E, and F.

It is also to be understood that the specific devices and processes illustrated in the attached drawings, and described in the following specification, are simply exemplary examples of the disclosure. Hence, specific dimensions and other physical characteristics related to the examples disclosed herein are not to be considered as limiting.

When used in relation to a component of a fluid injector system such as a fluid reservoir, a syringe, a fluid pump, or a fluid line, the term “distal” refers to a portion of said component nearest to a patient. When used in relation to a component of a fluid injector system such as a fluid reservoir, a syringe, a fluid pump, or a fluid line, the term “proximal” refers to a portion of said component nearest to the injector of the fluid injector system (i.e. the portion of said component farthest from the patient). When used in relation to a component of a fluid injector system such as a fluid reservoir, a syringe, a fluid pump, or a fluid line, the term “upstream” refers to a direction away from the patient and towards the injector of the fluid injector system. For example, if a first component is referred to as being “upstream” of a second component, the first component is located nearer to the injector than the second component is to the injector. When used in relation to a component of a fluid injector system such as a fluid reservoir, a syringe, a fluid pump, or a fluid line, the term “downstream” refers to a direction towards the patient and away from the injector of the fluid injector system. For example, if a first component is referred to as being “downstream” of a second component, the first component is located nearer to the patient than the second component is to the patient.

As used herein, the terms “capacitance” and “impedance” are used interchangeably to refer to a volumetric expansion of injector components, such as fluid reservoirs, syringes, fluid pumps fluid lines, and/or other components of a fluid injector system as a result of pressurized fluids with such components and/or uptake of mechanical slack by force applied to components. Capacitance and impedance may be due to high injection pressures, which may be on the order of 300 psi for certain computed tomography (CT) procedures and 1200 psi in some angiographic (CV) procedures, and may result in a volume of fluid held within a portion of a component in excess of the desired volume selected for the injection procedure or the resting volume of the component. Additionally, capacitance of various components can, if not properly accounted for, adversely affect the accuracy of pressure sensors of the fluid injector system because the volumetric expansion of components can cause an artificial drop in measured pressure of those components.

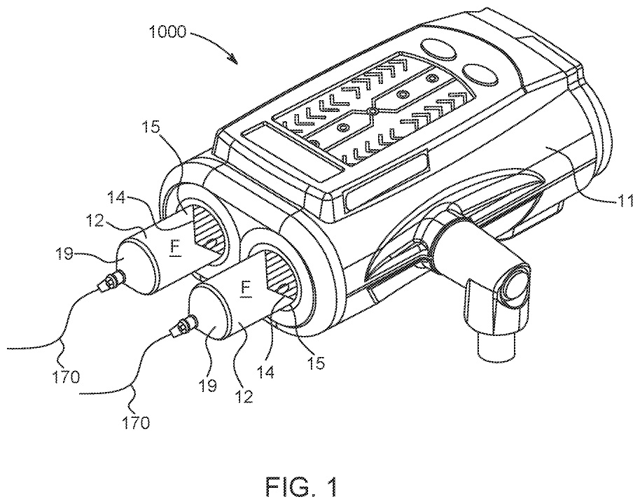

Referring to the drawings in which like reference characters refer to like parts throughout the several views thereof, the present disclosure is generally directed to valves, such as pinch valves, for regulating fluid flow in a fluid injector system. Referring first to FIGS. 1-3, examples of a fluid injector system 1000 in accordance with the present disclosure includes a housing 11 and at least one fluid reservoir, such as at least one syringe 12 or a fluid pump (not shown). The fluid injector system 1000 further includes a drive component to control fluid flow into or out of a fluid reservoir, such as a piston 13 associated with each of the syringes 12 that drives a plunger 14 within a barrel of the syringe 12. The at least one syringe 12 is generally adapted to releasably interface with the housing 11 at a syringe port 15. The fluid injector system 1000 is generally configured to deliver at least one fluid F to a patient during an injection procedure. The at least one syringe 12 of the fluid injector system 1000 is configured to be filled with at least one medical fluid F, such as an imaging contrast media, saline solution, or any desired medical fluid. Each syringe 12 may be filled with a different medical fluid F. The fluid injector system 1000 may be a multi-syringe injector, as shown, wherein several syringes 12 may be oriented side-by-side or in another spatial relationship and are separately actuated by respective pistons associated with the injector system 1000.

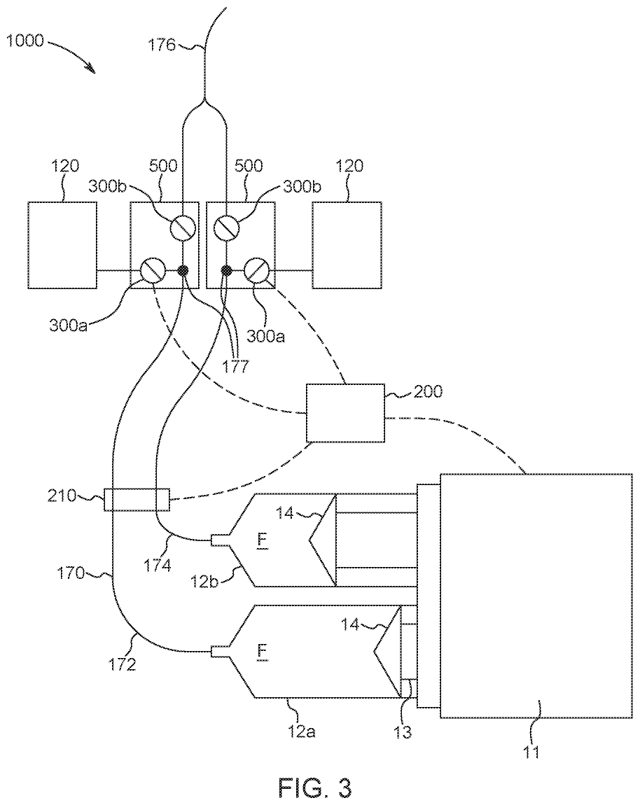

With continued reference to FIGS. 1-3, the fluid injector system 1000 may be used during a medical procedure to inject the at least one medical fluid F into the vasculature of a patient by driving the plungers 14 associated with the at least one syringe 12 with the at least one piston 13. The at least one piston 13 may be reciprocally operable upon the plunger 14. Upon engagement, the at least one piston 13 may move the plunger 14 toward a proximal end of the at least one syringe 12 to draw the medical fluid F into the at least one syringe 12 from a bulk fluid reservoir (see FIG. 3), such as a vial, bottle, or intravenous bag. The at least one piston 13 may further move the plunger 14 toward a distal end 19 of the at least one syringe 12 to expel the fluid F from the at least one syringe 12 during a priming, purging, or fluid delivery step. A fluid path set 170 may include at least one tube or tube set configured to be in fluid communication with each syringe 12 to place each syringe 12 in fluid communication with a flexible administration tube 176 for delivering the fluid F from each syringe 12 to a patient at a vascular access site.

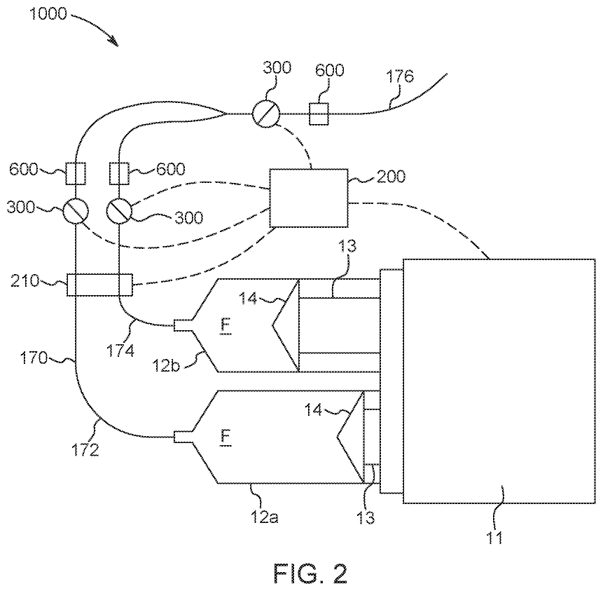

As shown in FIG. 2, the fluid path set 170 may include a first flexible tube 172 fluidly connected to a first syringe 12a and a second flexible tube 174 fluidly connected to a second syringe 12b. The first flexible tube 172 and the second flexible tube 174 of the fluid path set 170 may merge into the administration tube 176 for connection to the patient, or to one or more intervening components, such as a catheter, connected to the patient. Each of the first flexible tube 172, the second flexible tube 174, and the administration tube 176 of the fluid path set 170 may be formed of a flexible and reversibly compressible material, such as a polymer, and may contain one or more reinforcing materials, such as one or more braided fiber components. As used hereon, the term “reversibly compressible” means that the cross-sectional shape of the flexible tube of the fluid path set 170, or a portion thereof, can change by applying a force thereto, and upon release of the applied force, the flexible tube of the fluid path set 170 returns to its original shape. For example, a force may be applied to an outside surface of the flexible tube of the fluid path set 170, causing the diametrically opposed points on the inner sidewall of the flexible tube of the fluid path set 170 to be brought together to alter the cross-sectional area of a lumen of flexible tube of fluid path set 170.

With continued reference to FIGS. 2-3, the fluid injector system 1000 may further include a controller 200 for controlling actuation of the at least one piston 13 and other components of the fluid injector system 1000. The fluid injector system 1000 may perform one or more injection procedures according to one or more injection protocols stored in a memory of or accessible by the controller 200. The controller 200 may communicate with at least one air detector 210 configured to detect the presence of air in the fluid path set 170. The controller 200 may be configured to stop actuation of the at least one syringe 12a, 12b in response to the air detector 210 detecting air in the tubing of the fluid path set 170 in order to prevent air from being injected into the patient. Stopping actuation of the at least one syringe 12a, 12b may include halting distal movement of the piston 13 of the at least one syringe 12a, 12b and/or moving at least one valve, such as a rolling cam pinch valve of the present disclosure to a closed position wherein a portion of the downstream tubing is compressed to prevent fluid flow past the at least one valve.

Further details and examples of suitable non-limiting powered injector systems, including syringes, controllers, air detectors, and/or fluid path sets are described in U.S. Pat. Nos. 5,383,858; 7,553,294; 7,666,169; 8,945,051; 10,022,493; and 10,507,319, the disclosures of which are hereby incorporated by reference in their entireties.

The fluid injector system 1000 may further include one or more valves 300 disposed at various locations along the fluid path set 170. Each of the valves 300 may be in the form of a shut-off valve and/or a flow rate control valve to regulate flow of the medical fluid F to the patient. In the embodiment shown in FIG. 2, one of the valves 300 is provided on each of the first flexible tube 172, the second flexible tube 174, and the administration tube 176 of the fluid path set 170. In some embodiments, at least one valve 300 may be provided only on each of the first flexible tube 172 and the second flexible tube 174 of the fluid path set 17. In some embodiments, a valve 300 may be provided only on the administration tube 176 of the fluid path set 170. In some embodiments, one or more of the valves 300 may be mounted directly to the housing 11 or other component of the fluid injector system 1000.

Each of the valves 300 may be controllable by the controller 200 to regulate the flow of the fluid F through the fluid path set 170. For example, any or all of the valves 300 may be closed by the controller 200 in response to the at least one air detector 210 detecting air in the fluid path set 170. Closure of each valve 300 reversibly compresses the flexible tubing 172, 174, and/or 176 of the fluid path set 170 to halt fluid flow through the fluid path set 170. Closure of the valves 300 in this manner prevents the medical fluid F from advancing downstream of the valves 300, thereby preventing air from being injected into the patient due to relief of capacitance within the fluid path set 170 and/or the syringes 12a, 12b. In contrast, only halting the movement of the at least one piston 13 (without closing the valve or valves 300) may allow the medical fluid F, and any air contained therein, to be injected into the patient as capacitance in the fluid path set 170, the syringes 12 a, 12b, and/or the mechanical slack of the one or more components of the fluid injector system 1000 is relieved and the released volume of fluid F flows through the tubing.

Alternatively, one or more of the at least one valves 300 may be biased in the closed position such that in a resting state, the valve 300 prevents fluid flow through the corresponding flexible tubing 172, 174, and/or 176 of the fluid path set 170. In this embodiment, the controller 200 may regulate flow of fluid F through the fluid path set 170 by energizing and moving the valve 300 to the open position to allow fluid flow therethrough. Any or all of the valves 300 may be closed by the controller 200 in response to the at least one air detector 210 detecting air in the fluid path set 170 by removing any energizing power to the valve 300 such that the valve 300 returns to the biased closed position, thereby preventing the medical fluid F from advancing downstream of the valves 300, thereby preventing air from being injected into the patient due to relief of capacitance within the fluid path set 170 and/or the syringes 12a, 12b.

The valves 300 may alternatively or additionally be utilized to perform functions other than halting fluid flow in response to air detection. In some embodiments, the valve 300 provided on the first flexible tube 172 of the fluid path set 170 may be closed by the controller 200 to prevent backflow of pressurized medical fluid F from the second flexible tube 174 and/or second syringe 12 into the first flexible tube 172 or first syringe due to a difference in pressure and/or fluid viscosity between the two syringes and associated tubing. Similarly, a valve 300 on the second flexible tube 174 may be closed to prevent backflow from a higher pressurized fluid in the first flexible tube 172 and/or first syringe 12. In some embodiments, any or all of the valves 300 may be partially closed by the controller 200 to limit or control a flow rate of the medical fluid F in accordance with an injection protocol. For example, partially closing the valve 300 associated with the first flexible tube 172 may decrease the fluid flow of the first fluid through the first flexible tube 172. The reduction in fluid flow rate may be calculated according to an algorithm with variables associated with percent fluid path closure, fluid pressure, upstream tubing capacitance, fluid viscosity, pressure drop across the valve 300, and the like.

Referring in particular to FIG. 3, an embodiment of a fluid delivery system 1000 in accordance with the present disclosure is illustrated in which each syringe 12a, 12b is associated with a bulk fluid reservoir 120 and a valve assembly 500. Each valve assembly 500 includes two valves 300a, 300b for selectively controlling fluid flow between the syringes 12a, 12b and the associated bulk fluid reservoir 120, and the administration tube 176. Each of the valve assemblies 500 may be operatively associated with the controller 200 such that the controller 200 can actuate the valves 300a, 300b to regulate and control fluid flow. In particular, a first valve 300a may be associated with flexible tubing between the bulk fluid reservoir 120 and the valve 300a and each syringe 12a, 12b, while a second valve 300b may be associated with flexible tubing between each syringe 12a, 12b and the valve 300b and with the administration tube 176. During a fill operation in which the piston 13 is retracted to draw fluid F from the bulk reservoir 120 into at least one of the syringes 12a, 12b, the associated valve 300a may be opened by the controller 200 to allow fluid communication between the bulk reservoir 120 and the syringe 12a, 12b. The valve 300b may be closed to prevent fluid and/or air downstream of the valve 300b from being drawn into the syringe 12a, 12b during the fill operation.

During an injection procedure in which the piston 13 is extended to inject fluid F from the syringe 12 into the patient, the valve 300b may be open to allow fluid communication between the syringe 12 and the administration tube 176. The valve 300a may be closed to prevent fluid F from being injected into the bulk fluid reservoir 120.

The flexible tubing associated with the valve 300a and the valve 300b may merge at a junction 177 upstream of the valves 300a, 300b. As such, the flexible tubing between the junction 177 and the valve 300a is subjected to injection pressure, which may be up to approximately 1200 psi for some angiographic procedures. As such, the flexible tubing associated with the upstream side of the valve 300a must be able to withstand fluid pressures of at least 1200 psi. Further, all tubing between the syringes 12a, 12b and the valve 300b and with the administration tube 176 may also be subject to fluid pressures up to approximately 1200 psi and must therefore be able to withstand such pressure without leaking or failure.

In some embodiments, the valves 300a, 300b of each valve assembly 500 may be mutually actuated by the controller 200. For example, in some embodiments, both valves 300a, 300b may be actuated by a common motor, as discussed in greater detail herein.

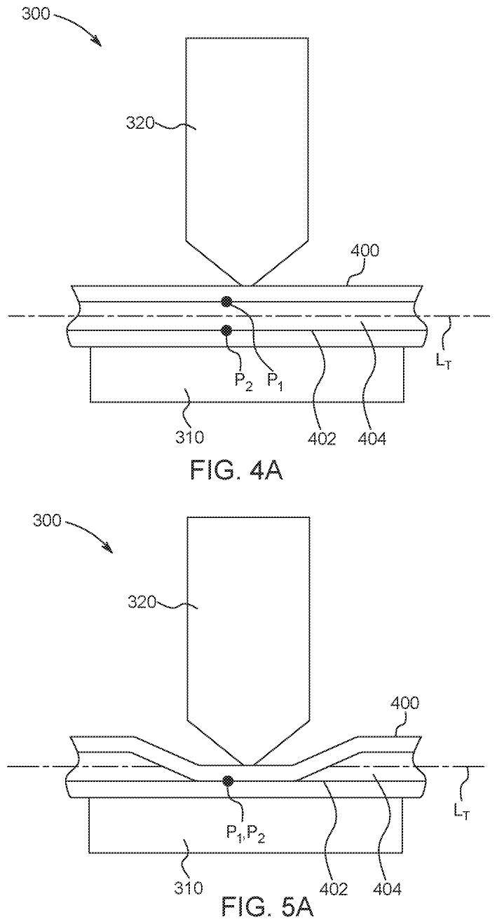

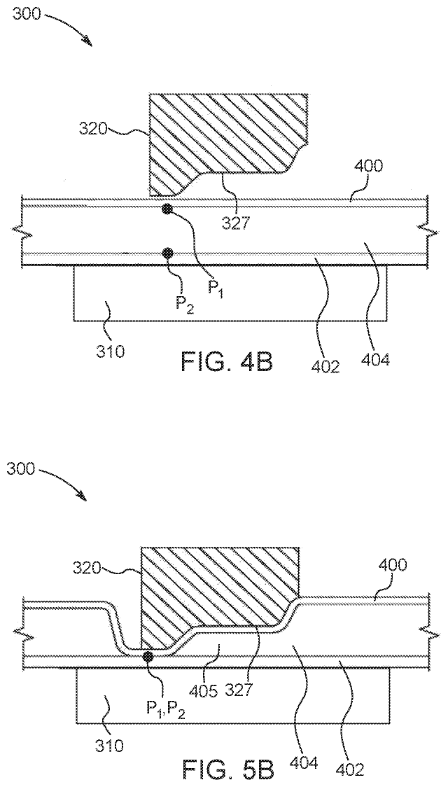

Having generally described embodiments of the fluid delivery system 1000, specific features of the valves 300, 300a, 300b (hereinafter referred to as “valve 300”) and the valve assemblies 500 will now be discussed. Each valve 300 may be in the form of a pinch valve configured to reversibly compress an associated flexible tube, as shown schematically in FIGS. 4A and 5A. Other types of valves such as stopcocks are also understood to be within the scope of the present disclosure. Referring now to FIGS. 4A and 5A, a schematic of a pinch valve 300 in an open position (FIG. 4A) and a closed position (FIG. 5A) is shown with associated flexible tubing 400. In the open position shown in FIG. 4A, an anvil 320 of the valve 300 is in a retracted position. In the closed position shown in FIG. 5A, the anvil 320 of the valve 300 is in an extended position to reversibly compresses the flexible tubing 400 against a backing plate 310 to close a lumen 404 of the flexible tubing 400. As used herein, the term “open”, when used in connection with the flexible tubing 400, means that an inner sidewall 402 of the flexible tubing 400 is substantially uncompressed, as shown in FIG. 4A, such that a cross sectional area of a lumen 404 of the flexible tubing 400 is at least the same diameter as in a natural, relaxed state or a larger diameter due to swelling under pressure (i.e., capacitance swelling). The term “fully closed” means that the flexible tubing 400 is reversibly compressed such that diametrically opposed points P₁, P₂ of the inner sidewall 402 are brought into contact with one another, thereby reducing the cross-sectional area of the lumen 404 to substantially zero, as shown in FIG. 5A. Fluid flow along the longitudinal axis LT of the flexible tubing is thus prohibited through the lumen 404. The term “closed” may be used interchangeably with the term “fully closed” herein. The terms “partially open” and “partially closed” mean that the flexible tubing 400 is reversibly compressed such that diametrically opposed points P₁, P₂ of the inner sidewall 402 are brought towards one another, reducing the cross-sectional area of the lumen 404 relative to the natural, relaxed state of the flexible tubing 400. However, the cross-sectional area of the lumen 404 when “partially open” and/or “partially closed” is greater than zero, thus allowing some fluid flow through the lumen 404 past the valve 300. In some embodiments, the controller 200 (see FIGS. 2 and 3) may be configured to move the anvil 320 and/or backing plate 310 to any position between the open and closed position to change the cross-sectional area of lumen 404, thereby controlling the flow rate of fluid through valve 300.

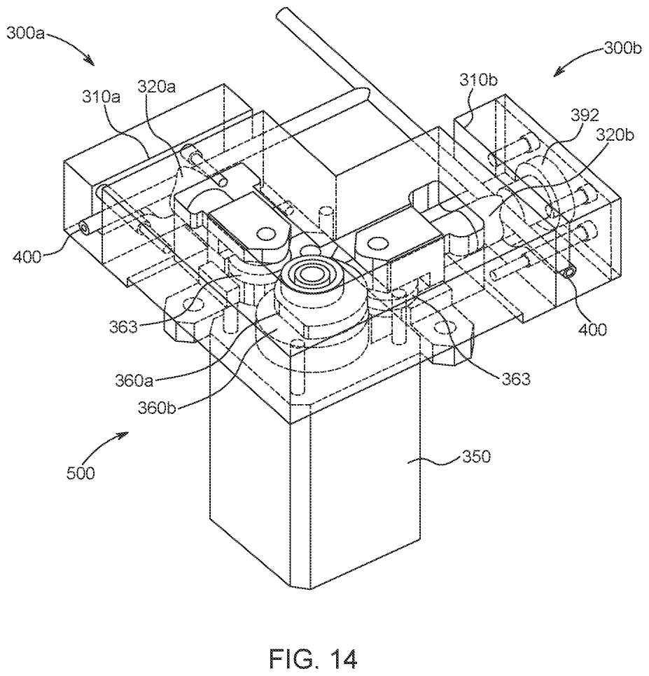

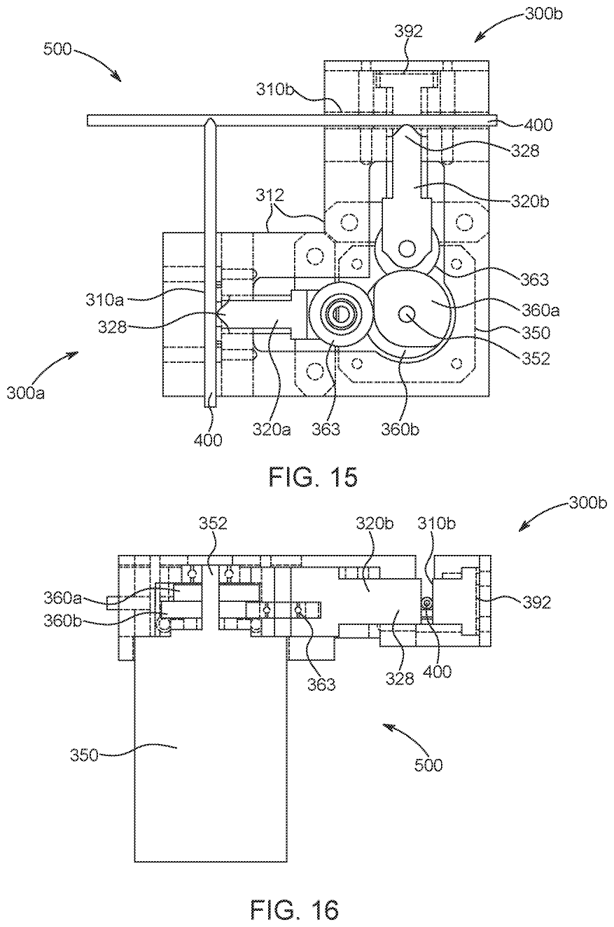

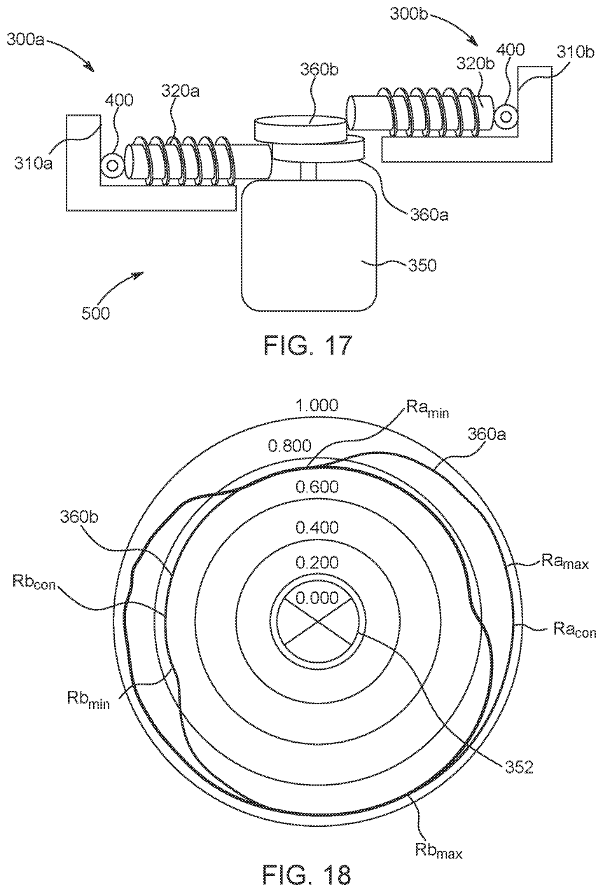

Referring now to FIGS. 4B and 5B, a schematic of a pinch valve 300 in an open position (FIG. 4B) and a closed position (FIG. 5B) is shown in accordance with an embodiment of the present disclosure. In this embodiment, the valve 300 is in the form of a high crack pressure valve configured to automatically open in response to a predetermined fluid pressure in the flexible tubing 400 In the open position shown in FIG. 4B, an anvil 320 of the valve 300 is in a retracted position. In the closed position shown in FIG. 5B, the anvil 320 of the valve 300 is in an extended position to reversibly compresses the flexible tubing 400 against a backing plate 310 to close a lumen 404 of the flexible tubing 400. A tip profile of the anvil 320 defines at least one step 327. As shown in FIG. 5B, when the lumen 404 is fully compressed at points P₁, P₂, a partially compressed region 405 of the lumen 404 is present in the vicinity of the step 327. Fluid pressure within the partially compressed region 405 acts against the step 327 and/or the backing plate 310, and, if there is sufficient fluid pressure in the partially compressed region 405, the anvil 320 is forced back to the open position of FIG. 4B or to a partially open position. Thus, the valve 300 may serve as a crack pressure valve configured to open at a predetermined fluid pressure in the lumen 404. In some embodiments, as described herein with reference to FIGS. 14-18, the backing plate 310 may be movable such that fluid pressure in the partially compressed region 405 of the lumen 404 acts against the step portion 327 and the backing plate 327 to force the backing plate away from the anvil 320, thereby opening the valve 300. In some embodiments, as described herein with reference to FIGS. 14-18, the backing plate 310 may include an adjustable biasing member 392 such that the fluid pressure necessary to open the valve 300 may be set to a predetermined value.

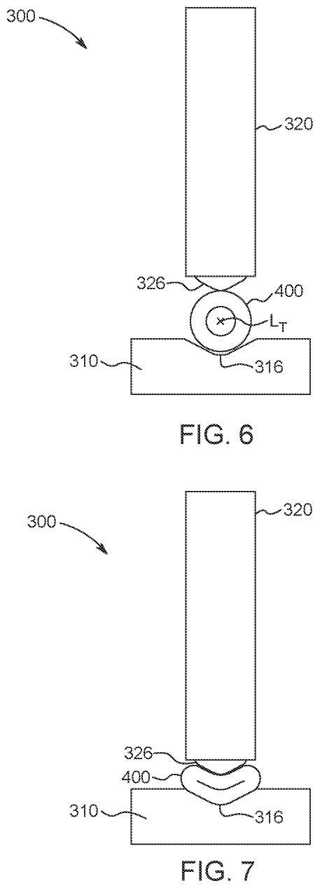

Referring now to FIGS. 6 and 7, in some embodiments, the backing plate 310, may include a groove or channel 316 for indexing and/or retaining the flexible tubing 400 in a particular position relative to the anvil 320. In certain embodiments, the groove or channel 316 may be arcuate and have a radius substantially equal to or greater than an outer diameter of the flexible tubing 400. The anvil 320 may include a projection 326 approximately corresponding to the shape of the groove or channel 316. In addition to indexing the flexible tubing 400, the groove or channel 316 induces the flexible tubing 400 to compress in an approximate U-shape or V-shape, as shown in FIG. 7, which may reduce the force required to reversibly compress the flexible tubing 400 and more effectively closes the lumen 404 during compression of the flexible tubing 400. The groove or channel 316 may also reduce the “dog-bone” effect as the flexible tubing 400 is compressed. As used herein, the term “dog-bone effect” is a phenomenon where the compressed cross-section of the lumen 404 of the flexible tubing has a shape of a dog-bone (i.e., not fully compressed at the lateral sides of the lumen) due to increased material thickness at the lateral sides of the compressed flexible tubing 400 relative to the interior of the compressed flexible tubing 400.

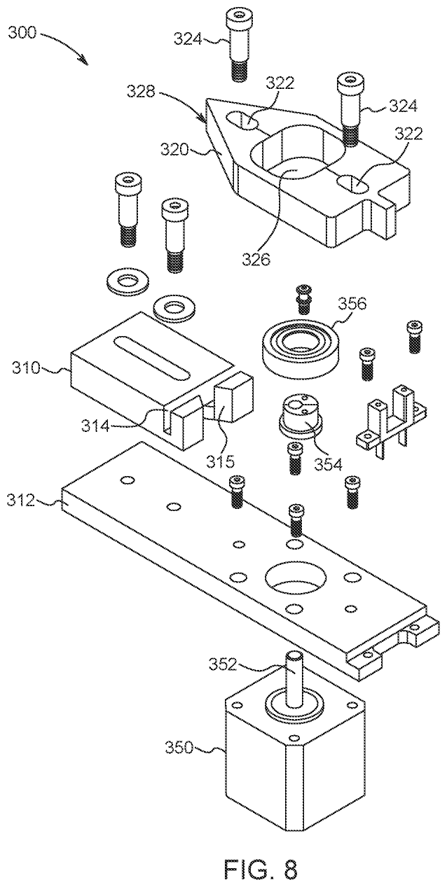

Referring now to FIG. 8, a pinch valve 300 is shown in accordance with an embodiment of the present disclosure. In this embodiment of the valve 300, the backing plate 310 is rigidly mounted to a frame 312. A motor 350 is also rigidly mounted to the frame 312 and includes a shaft 352 coupled to a cam 354. A central axis of the cam 354 is offset from a rotational axis of the shaft 352, such that as the shaft 352 rotates, the cam 354 rotates eccentrically relative to the shaft 352. A bearing 356, for example a roller bearing or bushing, is mounted to the perimeter of the cam 354. The anvil 320 is mounted to the frame 312 in a manner that permits the anvil 320 to slide relative to the backing plate 310. In some embodiments, the anvil 320 includes one or more slots 322 which allow the anvil 320 to slide relative to guide pins or bolts 324 secured to the frame 312. The anvil 320 further includes a bearing cavity 326 into which the bearing 356 and the cam 354 are disposed. As the cam 354 rotates, the bearing 356 engages the bearing cavity 326 to slide the anvil 320 along the frame 312, towards or away from the backing plate 310.

With continued reference to FIG. 8, the backing plate 310 may include a slot 314 into which the flexible tube 400 (shown in FIGS. 4A-7) may be secured. The slot 314 may include an opening 315 facing the anvil 320 such that a tip 328 of the anvil 320 can engage and reversibly compress the flexible tubing 400 through the opening 315, as shown in FIGS. 4A-7. The motor 350 may be in operative communication with the controller 200 (see FIGS. 2 and 3) such that the controller 200 actuates the motor 350 to slide that anvil 320 to a desired position to open, close, partially open, or partially close the flexible tubing 400 (shown in FIGS. 4A-7). In some embodiments, the motor 350 may be a stepper motor, a solenoid, or other conventional electromechanical motor. In some embodiments, the motor 350 may be capable of closing the flexible tubing 400 in approximately 0.25 seconds or less.

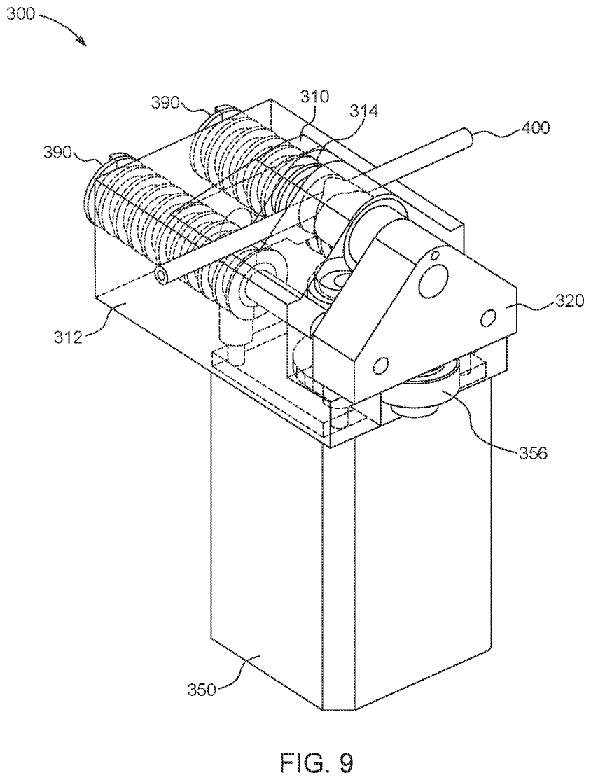

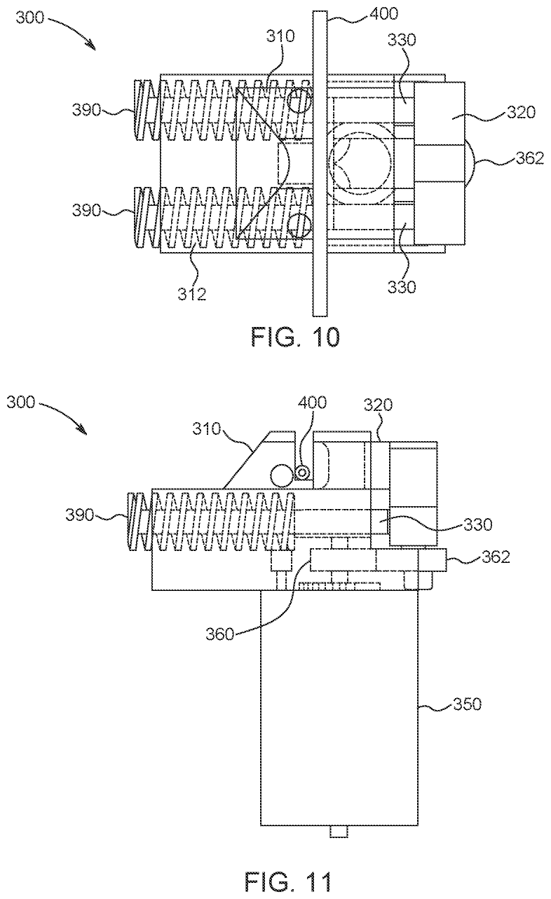

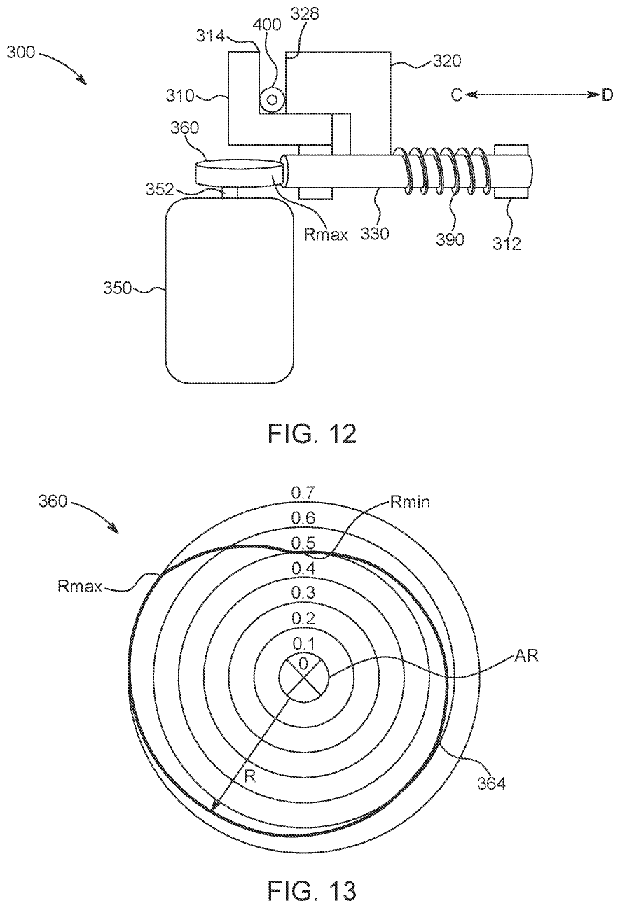

Referring now to FIGS. 9-13, a pinch valve 300 is shown in accordance with another embodiment of the present disclosure. In this embodiment of the valve 300, the backing plate 310 and the motor 350 may be rigidly is mounted to the frame 312. An eccentric cam 360 is coupled to the shaft 352 of the motor 350 and is configured to drive the anvil 320 relative to the frame 312. In some embodiments, the eccentric cam 360 may interface directly with the anvil 320 (see, e.g. FIG. 12), while in other embodiments, the eccentric cam 360 may engage a bearing 362 mounted to the anvil 320 (see FIGS. 9-11).

Referring specifically to FIG. 13, the eccentric cam 360 has an outer profile 364 having an irregular radius R about a rotational axis AR of the motor shaft 352. As the motor shaft 352 rotates the cam 360, a different portion of the outer profile 364 engages the bearing 362 and/or the anvil 320 to move the anvil 320 relative to the frame 312. When the cam 360 is oriented such that a portion of maximum radius Rₘₐₓ engages the bearing 362 and/or the anvil 320 (as shown in FIG. 12), the anvil 320 is forced to a maximum position in a direction D away from the flexible tubing 400. In this position, the flexible tubing 400 is substantially uncompressed (as shown in FIGS. 4A and 12) with the valve 300 in the open position. Conversely, when the cam 360 is oriented such that a portion of minimum radius Rₘᵢₙ engages the bearing 362 and/or the anvil 320, the anvil 320 may move to a maximum position in the direction C toward the flexible tubing 400. In this position, the tip 328 of the anvil 320 engages and fully compresses the flexible tubing 400 (as shown in FIGS. 5A and 11) such that the valve 300 is in the closed position. The outer profile 364 of the cam 360 may be selected and/or designed to reduce the torque demand on the motor 350 during opening/closing of the valve 300, to decrease the time required for moving the valve 300 between the open and closed positions, or to optimize various other performance characteristics of the valve 300.

With continued reference to FIG. 9-13, the anvil 320 may include one or more connecting rods 330 that slide within the frame 312 and are biased relative to the frame 312 by one or more biasing elements 390, such as one or more springs. In some embodiments, the biasing elements 390 bias the anvil 320 in the direction C toward the flexible tubing 400, such that in absence of power from the motor 350, the anvil 320 is biased to close the flexible tubing 400. As the anvil 320 is moved in the direction D, the biasing elements 390 act against the anvil 320, such that the force of the biasing elements 390 must be overcome by the motor 350 to open the flexible tubing 400. Because the biasing elements 390 bias the anvil 320 toward the flexible tubing 400 and the closed position, the valve 300 of FIGS. 9-13 is capable of rapidly compressing the flexible tubing 400 when the motor 350 is actuated by the controller 200 (see FIGS. 2-3), or when power to the system 1000 interrupted. In addition, the pressure exerted by the biasing elements 390 takes up any variation in tubing diameter due to stress relaxation and/or manufacturing tolerance of the flexible tubing 400, ensuring full closure of the flexible tubing 400. More particularly, the outer profile 364 of the cam 360 may be configured such that in the absence of the flexible tubing 400 in the slot 314, the tip 328 of anvil 320 can travel in the direction C closer to the backing plate 310 than the compressed thickness of the flexible tubing 400. This overtravel in the anvil 320 is then absorbed by the biasing elements 390 when the flexible tubing 400 is compressed, such that the anvil 320 only travels in the direction C a sufficient distance to compress the flexible tubing 400 into the closed position while not over-compressing and damaging the flexible tubing 400.

In some embodiments, the biasing elements 390 may be selected or configured to provide sufficient pressure to reversibly compress the flexible tubing 400 without damaging the flexible tubing 400. In some embodiments, the biasing elements 390 may be adjustable and the pressure provided by the biasing elements 390 may be set based on a measured or estimated fluid pressure within the flexible tubing 400. For example, in an embodiment, the pressure provided by the biasing elements 390 may be adjusted in real time by the controller 200 in response to fluid pressure changes in the flexible tubing 400, by the properties (e.g., wall thickness, tubing material, inner diameter, outer diameter) of the flexible tubing 400, the type of medical fluid F in the tubing, the fluid pressure (e.g., as measured by the motor current or load on the injector piston motor or the valve motor) and/or the various flow rates required during specific times of the programmed fluid injection protocol. For example, because. For example, because the fluid pressure within the flexible tubing 400 may be relatively low, e.g. 400 psi, during a substantial portion of the usage of the system 1000, the pressure provided by the biasing elements 390 may be set to a relatively low value during the “low” pressure portions of an injection procedure, to increase the lifespan of the flexible tubing 400, prevent changes in tube wall thickness or inner diameter of the portion of the flexible tubing 400 contacting the valve elements, and/or impact compressibility or rebound of the portion of the flexible tubing 400 contacting the valve elements. The pressure provided by the biasing elements 390 may be increased by the controller 200 as the fluid pressure and/or the programmed flow rate in the flexible tubing 400 increases to ensure that high fluid pressure does not overcome the force of the biasing elements 390 and inadvertently open flexible tubing 400 or allow fluid to leak through valve 300. Adjustment of the force of the one or more biasing elements 390 may be affected, for example, by a second electromechanical motor or other electromotive force to compress biasing elements 390. Examples of the one or more biasing elements 390 may include a spring, such as a conventional spring or spring with an adjustable spring force constant.

In some embodiments, the cam 360 may be coupled to the motor 350 by a clutch and/or freewheel mechanism that locks the cam 360 to the shaft 352 when power is supplied to the motor 350, such that the cam 360 rotates with the motor shaft 352. The clutch or freewheel mechanism may disengage when power to the motor 350 is interrupted such that the cam 360 is allowed to rotate relative to the shaft 352. In particular, the cam 360 may be biased (for example by a torsion spring) such that when the clutch and/or freewheel mechanism disengages in the absence of power to the motor 350, the cam 360 rotates independently of the shaft 352 to the closed position of the valve 300. As such, the portion of minimum radius Rₘᵢₙ is oriented toward the anvil 320, thereby allowing anvil 320 advance toward backing plate 310 under the influence of the one or more biasing elements 390 to compress flexible tubing 400.

The force exerted by the biasing elements 390 and the mass of the anvil 320 may be selected to optimize closing time of the valve 300. In particular, a high force exerted by the biasing elements 390 and low mass of the anvil 320 may be utilized to minimize the time required to compress the flexible tubing 400 in response to receiving a signal from the controller 200.

In some embodiments, the one or more biasing elements 390 may be oriented to bias to valve 300 toward the open position in which the flexible tubing 400 is substantially uncompressed. In some embodiments, the cam 360 may be coupled to the motor 350 by a clutch and/or freewheel mechanism that locks the cam 360 to the shaft 352 when power is supplied to the motor 350, such that the cam 360 rotates with the motor shaft 352. The clutch or freewheel mechanism may disengage when power to the motor 350 is interrupted such that the cam 360 is allowed to rotate relative to the shaft 352. In particular, the cam 360 may be biased (for example by a torsion spring) such that when the clutch and/or freewheel mechanism disengages in the absence of power to the motor 350, the cam 360 rotates independently of the shaft 352 to the open position of the valve 300. As such, the portion of maximum radius Rₘₐₓ is oriented toward the anvil 320, thereby forcing the anvil 320 away from the backing plate 310 to uncompress the flexible tubing 400.

Referring now to FIGS. 14-18, a valve assembly 500 is shown in accordance with another embodiment of the present disclosure. The valve assembly 500 of FIGS. 14-18 may correspond to either of the valve assemblies 500 shown and described with reference to FIG. 3. In particular, the valve assembly 500 of FIGS. 14-18 includes two pinch valves 300a, 300b actuated by a common motor 350 controlled by the controller 200 (see FIG. 3). Each pinch valve 300a, 300b respectively includes an anvil 320a, 320b driven towards a backing plate 310a, 310b by one of two cam 360a, 360b of a dual cam assembly 360, similar to the embodiment shown in FIGS. 9-13. The cams 360a, 360b associated with the pinch valves 300a, 300b are coupled to the motor shaft 352 of the common motor 350 for operating the pinch valves 300a, 300b, and thus both cams 360a, 360b share the rotation axis of the motor shaft 352. The cams 360a, 360b associates with each pinch valve 300a, 300b engage the respective anvils 320a, 320b to drive the anvils 320a, 320b towards the corresponding backing plate 310a, 310b as the motor shaft 352 is rotated. In some embodiments, as shown in FIGS. 14-16, each anvil 320a, 320b may include a cam follower bearing 363 that is engaged by the associated cam 360a, 360b to drive the anvil 320, reducing wear on the anvils 320a, 320b and the cams 360a, 360b. The backing plate 310a, 310b associated with one or both of the pinch valves 300a, 300b may include a biasing element 392, such as a wave washer, Belleville washer, or spring, which biases the backing plate 310a, 310b towards the respective anvil 320a, 320b. The biasing element 392 thus preloads the flexible tubing 400 to account for stress relaxation and/or manufacturing tolerance of the flexible tubing 400, thereby ensuring the desired closing force is applied by the anvil 320a, 320b. In some embodiments, the biasing element 392 may be adjustable and the spring rate may be set to account for compliance in the flexible tubing 400. In certain embodiments, one of the pinch valves (i.e., pinch valve 300b associated with the flexible tubing 400 between the syringe 12 and the patient (i.e., tubing 170 and 176 in FIG. 3) may include a biasing element 392, while the other pinch valve (i.e., pinch valve 300a associated with the bulk fluid reservoir 120) may not include a biasing element 392.

In some embodiments, the biasing element 392 may be adjustable and the pressure provided by the biasing element 392 may be set based on a measured or estimated fluid pressure within the flexible tubing 400. For example, in an embodiment, the pressure provided by the biasing element 392 may be adjusted in real time by the controller 200 in response to fluid pressure changes in the flexible tubing 400, by the properties (e.g., wall thickness, tubing material, inner diameter, outer diameter) of the flexible tubing 400, the type of medical fluid F in the tubing, the fluid pressure (e.g., as measured by the motor current or load on the injector piston motor or the valve motor) and/or the various flow rates required during specific times of the programmed fluid injection protocol. For example, because the fluid pressure within the flexible tubing 400 may be relatively low, e.g. 400 psi, during a substantial portion of the usage of the system 1000, the pressure provided by the biasing element 392 may be set to a relatively low value during the “low” pressure portions of an injection procedure, to increase the lifespan of the flexible tubing 400, prevent changes in tube wall thickness or inner diameter of the portion of the flexible tubing 400 contacting the valve elements, and/or impact compressibility or rebound of the portion of the flexible tubing 400 contacting the valve elements. The pressure provided by the biasing element 392 may be increased by the controller 200 as the fluid pressure and/or programmed flow rate in the flexible tubing 400 increases to ensure that high fluid pressure does not overcome the force of the biasing element 392 and inadvertently open the flexible tubing 400 or allow fluid to leak through the valve 300. Adjustment of the force of the one or more biasing elements 390 may be affected, for example, by a second electromechanical motor or other electromotive force to compress the biasing elements 390. Suitable examples of the one or more biasing elements may include a spring, such as a conventional spring or a spring with an adjustable spring force constant.

In some embodiments, the spring rate of the biasing element 392 may be set to function as a high crack pressure valve. In particular, the biasing element 392 may be configured to compress at a predetermined pressure, causing the backing plate 310a, 310b to be displaced away from the anvil 320a, 320b and thereby open the flexible tubing 400 when a predetermined fluid pressure is present in the flexible tubing 400, wherein the pressure of the fluid in the tubing downstream from the valve 300a, 300b does not impact the pressure at which the valve 300a, 300b opens (“cracks”), see, for example U.S. Published Application No. 2016/0030662, the disclosure of which is hereby incorporated by reference in its entirety. In some embodiments the anvil 320a, 320b and/or the backing plate 310a, 310b may have a profile including a step 327 as shown in FIGS. 4B and 5B, such that the valve 300a and/or the valve 300b can serve as a high crack pressure valve as described herein with reference to FIGS. 4B and 5B.

As shown in FIG. 18, the cams 360a, 360b associated with the pinch valves 300a, 300b are clocked on the motor shaft 352 to achieve a desired relative timing of the opening/closing of the pinch valves 300a, 300b. Similar to the embodiment of the cam 360 shown in FIG. 13, each cam 360a, 360b may include an irregular radius to drive the corresponding anvils 320a, 320b. In particular, each cam 360a, 360b may include a respective portion of maximum radius Raₘₐₓ, Rbₘₐₓ and a respective portion of minimum radius Raₘᵢₙ, Rbₘᵢₙ. When the portion of the cam 360a, 360b nears and reaches the maximum radius Raₘₐₓ, Rbₘₐₓ during engagement with the cam follower bearing 363 and/or the anvil 320a, 320b, the anvil 320a, 320b is driven toward and reaches the maximum distance against the backing plate 310 to fully compress the associated flexible tubing 400. Conversely, when the portion of the cam 360a, 360b nears and reaches the minimum radius Raₘᵢₙ, Rbₘᵢₙ during engagement with the cam follower bearing 363 and/or the anvil 320a, 320b, the anvil 320a, 320b is driven away from the backing plate 310a, 310b such that the flexible tubing 400 is substantially uncompressed in an open position. Note that due to the differences in structure and arrangement of the anvils 320a, 320b of the embodiment of FIGS. 14-18 as compared to the embodiment of FIGS. 9-13, the portion of maximum radius Raₘₐₓ, Rbₘₐₓ of the cams 360a, 360b compresses the flexible tubing 400, whereas in the embodiment of FIGS. 9-13, the portion of minimum radius Rₘᵢₙ of the cam 360 compresses the flexible tubing 400. However, in certain embodiments, the anvils 320a, 320b and cams 360a, 360b may be arranged in a manner similar to that of the embodiment of FIGS. 9-13, such that the portion of minimum radius Raₘᵢₙ, Rbₘᵢₙ of cams 360a, 360b compresses flexible tubing 400 in the same way that a portion of minimum radius Rₘᵢₙ of cam 360 compresses flexible tubing 400 in embodiment of FIGS. 9-13.

With continued reference to FIG. 18, in some embodiments, one or both of the cams 360a, 360b may include a constant radius section Ra_{con}, Rb_{con}. Rotation of the motor shaft 352 within a span of the constant radius section Ra_{con}, Rb_{con} does not change the position of the corresponding anvil 320 because the radius over the span of the constant radius section Ra_{con}, Rb_{con} does not change. The cams 360a, 360b may be indexed relative to one another on the motor shaft 352 such that one of the cams 360a drives its corresponding anvil 320a due to a change in radius of that cam 360a, while the other of the cams 360b engages its corresponding anvil 320b with the constant radius section Rb_{con} so as to not move the corresponding anvil 320. The span of constant radius section Ra_{con}, Rb_{con} may coincide with the portion of maximum radius Raₘₐₓ, Rbₘₐₓ such that the anvils 320a, 320b compress the flexible tubing 400 for a portion of the rotation of the shaft 352 corresponding to the span of constant radius section Ra_{con}, Rb_{con}. As illustrated in FIG. 18, the profiles of the two cams 360a, 360b may be different from one another to achieve a desired timing for the opening and closing of the respective pinch valves 300a, 300b. In certain embodiments, the profiles of the cams 360a, 360b may be selected and/or designed to reduce the torque demand on the motor 350 during opening/closing of the valves 300a, 300b; to decrease the time required for moving the valves 300a, 300b between the open and closed positions; and/or to optimize various other performance characteristics of the valves 300a, 300b.

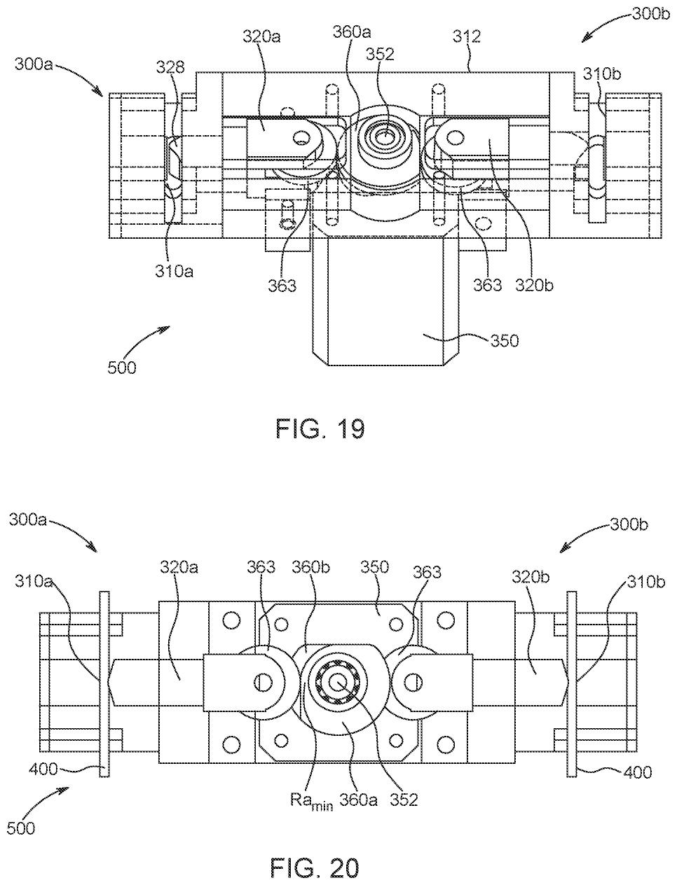

As shown in FIGS. 14-16, in certain embodiments, the anvils 320 of the pinch valves 300a, 300b may be oriented at approximately 90° relative to one another, such that the anvil 320a of the valve 300a moves along an axis approximately 90° relative to an axis along which the anvil 320b of the valve 300b moves. FIG. 19 shows an alternate embodiment of the valve assembly 500 in which the anvils 320 of the pinch valves 300a, 300b are oriented at approximately 180° relative to one another, such that the anvil 320a of the valve 300a moves along an axis approximately 180° relative to an axis along which the anvil 320b of the valve 300b moves. In other embodiments, the anvils 320a, 320b of the pinch valves 300a, 300b may be oriented at approximately 120° relative to one another. When two cams 360a, 360b are used, as in the embodiments of FIGS. 14-19, the pinch valves 300a, 300b may be oriented relative to one another at substantially any angle that does not result in physical interference of the various components.

The cams 360a, 360b may be indexed on the motor shaft 352 relative to one another to achieve the desired relative timing of the opening/closing of the pinch valves 300a, 300b according to the angular orientation of the of the pinch valves 300a, 300b. In some embodiments, as shown in FIG. 18, the cams 360a, 360b may be indexed relative to one another such that the portions of maximum radius Raₘₐₓ, Rbₘₐₓ of the cams 360a, 360b are located at approximately 90° relative to one another, corresponding to the angle between the axes of the anvils 320. As such, the portions of maximum radius Raₘₐₓ, Rbₘₐₓ of both cams 360a, 360b may simultaneously engage the respective anvils 320a, 320b, causing both valves 300a, 300b to be closed at the same time. Similarly, portions of minimum radius Raₘᵢₙ, Rbₘᵢₙ of the cams 360a, 360b are located at approximately 90° relative to one another, corresponding to the angle between the axes of the anvils 320a, 320b. As such, the portions of minimum radius Raₘᵢₙ, Rbₘᵢₙ of both cams 360a, 360b may simultaneously engage the respective anvils 320a, 320b, causing both valves 300a, 300b to be open at the same time. In other orientations of the cams 360a, 360b, the portion of minimum radius Raₘᵢₙ of the cam 360a engages the anvil 320a, while the portion of maximum radius Rbₘₐₓ of the cam 360b engages the anvil 320b. As such, the valve 300a is open at the time the valve 300b is closed. In other orientations of the cams 360a, 360b, the portion of maximum radius Raₘₐₓ of the cam 360a engages the anvil 320a, while the portion of minimum radius Rbₘᵢₙ of the cam 360b engages the anvil 320b. As such, the valve 300a is closed at the time the valve 300b is open. In other orientation of the cams 360a, 360b, portions between the minimum radius Raₘᵢₙ Rbₘᵢₙ and the maximum radius Raₘₐₓ Rbₘₐₓ may engage the corresponding anvils 320a, 320b to partially compress the flexible tubing 400.

Referring now to FIGS. 20-23, the valve assembly 500 of FIG. 19 is shown in four positions corresponding to various states of the valves 300a, 300b. The various states of the valves 300a, 300b shown in FIGS. 20-23 correspond to various rotational positions of the motor shaft 352 and the cams 360a, 360b coupled thereto. Referring first to FIG. 20, the motor shaft 352 and the cams 360a, 360b are rotated such that both valves 300a, 300b are open. That is, the motor shaft 352 and the cams 360a, 360b are rotated such that the portions of minimum radius Raₘᵢₙ, Rbₘᵢₙ of both cams 360a, 360b engage their respective anvils 320. (Note that the portion of minimum radius Rbₘᵢₙ of the cam 360b is obstructed from view in FIG. 20 by the cam 360a). Engagement with the portions of minimum radius Raₘᵢₙ, Rbₘᵢₙ of the cams 360a, 360b causes both anvils 320a, 320b to retract away from the associated flexible tubing 400 and therefore the flexible tubing 400 associated with each valve 300a, 300b is substantially uncompressed. Thus, fluid may flow through the flexible tubing 400 associated with each of the valves 300a, 300b.

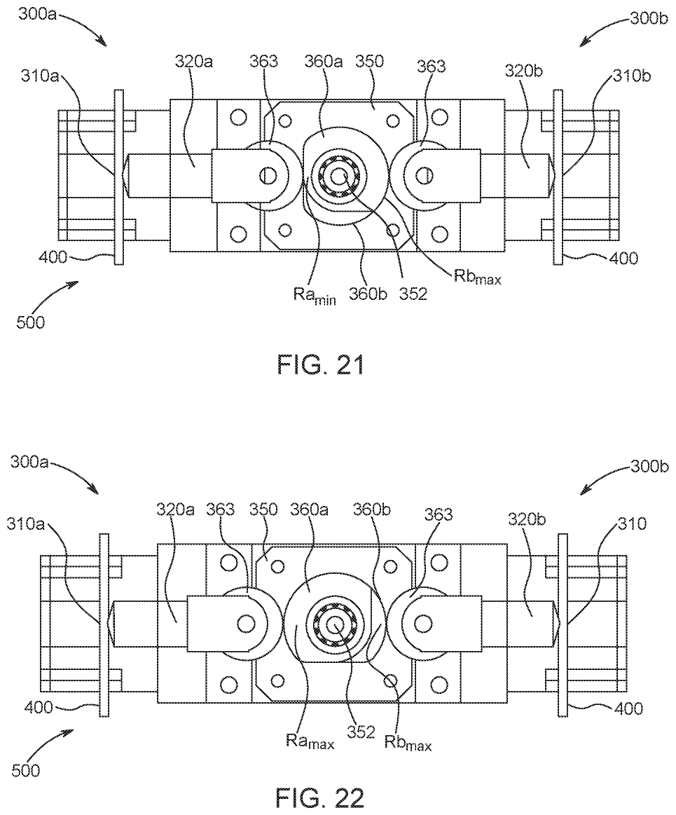

Referring next to FIG. 21, the motor shaft 352 and the cams 360a, 360b are rotated such that a first valve 300a is open and a second valve 300b is closed. That is, the motor shaft 352 and the cams 360a, 360b are rotated such that the portion of minimum radius Raₘᵢₙ of the cam 360a engages the anvil 320a associated with the valve 300a, whereas the portion of maximum radius Rbₘₐₓ of the cam 360b engages the anvil 320b associated with the valve 300b. The anvil 320a engaging the portion of minimum radius Raₘᵢₙ of the cam 360a is retracted away from the associated flexible tubing 400, and therefore the flexible tubing 400 associated with the first valve 300a is substantially uncompressed. The anvil 320b engaging the portion of maximum radius Rbₘₐₓ of the cam 360b is extended toward and compresses the flexible tubing 400 associated with the second valve 300b. Thus, fluid may flow through the flexible tubing 400 associated with the first valve 300a, but fluid may not flow through the flexible tubing 400 associated with the second valve 300b. The controller 200 (see FIG. 3) may be configured to move the valve assembly 500 to the position shown in FIG. 21 during a fill operation. As shown in FIG. 3, the first valve 300a may be associated with the bulk fluid reservoir 120 and may be open to allow fluid from the fluid reservoir 120 to be drawn into the syringe 12. The second valve 300b may be closed to prevent fluid and/or air downstream of the valve 300b from being drawn into the syringe 12 during the fill operation.

Referring next to FIG. 22, the motor shaft 352 and the cams 360a, 360b are rotated such that both valves 300a, 300b are closed. That is, the motor shaft 352 and the cams 360a, 360b are rotated such that the portions of maximum radius Raₘₐₓ, Rbₘₐₓ of the cams 360a, 360b engage the respective anvils 320 320 associated with both valves 300a, 300b. Engagement with the portions of maximum radius Raₘₐₓ, Rbₘₐₓ of the cams 360a, 360b causes both anvils 320a, 320b to extend toward and compress the associated flexible tubing 400. Thus, fluid may not flow through the flexible tubing 400 associated with either of the valves 300a, 300b. The controller 200 (see FIG. 3) may be configured to move the valve assembly 500 to the position shown in FIG. 22 to stop all fluid flow downstream of the valve assembly 500. For example, the controller 200 may move the valve assembly 500 to the position shown in FIG. 22 in response to the at least one air detector 210 (see FIG. 3) detecting one or more air bubbles in the syringe 12 or the portion of tubing associated with the at least one air detector 210. Closure of the valves 300a, 300b as shown in FIG. 22 may prevent the one or more air bubbles detected by the at least one air detector 210 from being injected into the patient.

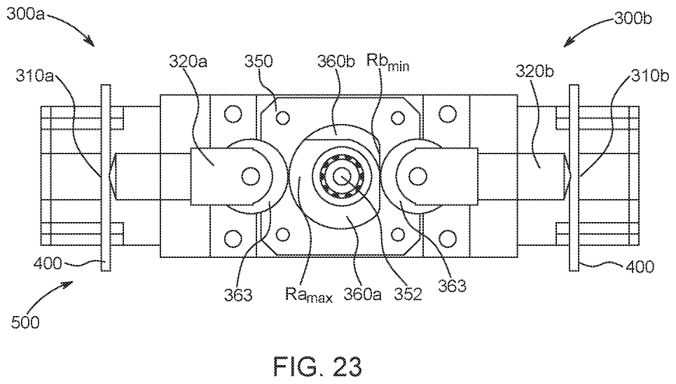

Referring next to FIG. 23, the motor shaft 352 and the cams 360a, 360b are rotated such that a first valve 300a is closed and a second valve 300b is open. That is, the motor shaft 352 and the cams 360a, 360b are rotated such that the portion of maximum radius Raₘₐₓ of the cam 360a engages the anvil 320a associated with the valve 300a, whereas the portion of minimum radius Rbₘᵢₙ of the cam 360b engages the anvil 320b associated with the valve 300b. The anvil 320 engaging the portion of maximum radius Raₘₐₓ of the cam 360a is extended toward and compresses the flexible tubing 400 associated with the first valve 300a. The anvil 320 engaging the portion of minimum radius Rbₘᵢₙ of the cam 360b is retracted away from the associated flexible tubing 400, and therefore the flexible tubing 400 associated with the second valve 300b is substantially uncompressed. Thus, fluid may flow through the flexible tubing 400 associated with the second valve 300b, but fluid may not flow through the flexible tubing 400 associated with the first valve 300a. The controller 200 (see FIG. 3) may be configured to move the valve assembly 500 to the position shown in FIG. 23 during an injection procedure. As shown in FIG. 3, the first valve 300a may be associated with the bulk fluid reservoir 120 and may be closed to prevent fluid from being injected into the fluid reservoir 120. The second valve 300b may be open to allow fluid to flow to the administration tube 176 and ultimately to the patient.

In some embodiments, the cams 360a, 360b may have a profile such that rotation of motor shaft 352 by approximately 90° moves the valve assembly 500 from the position shown in FIG. 20 (i.e., valve 300a open position, valve 300b open position) to the position shown in FIG. 21 (i.e., valve 300a open position, valve 300b closed position); a further rotation of the motor shaft 352 by approximately 90° moves the valve assembly 500 from the position shown in FIG. 21 to the position shown in FIG. 22 (i.e., valve 300a closed position, valve 300b open position); a further rotation of the motor shaft 352 by approximately 90° moves the valve assembly 500 from the position shown in FIG. 22 to the position shown in FIG. 23 (i.e., valve 300a closed position, valve 300b open position); and a further rotation of the motor shaft 352 by approximately 90° moves the valve assembly 500 from the position shown in FIG. 23 to the position shown in FIG. 20. The motor shaft 352 may also be rotated in the reverse order, i.e. from the position shown in FIG. 20 to the position shown in FIG. 23; from the position shown in FIG. 23 to the position shown in FIG. 22; from the position shown in FIG. 22 to the position shown in FIG. 21; and from the position shown in FIG. 21 to the position shown in FIG. 20. Alternatively, the motor shaft 352 may also be rotated in any desired direction to reach the desired valve configuration required for a specific portion of the injection procedure.

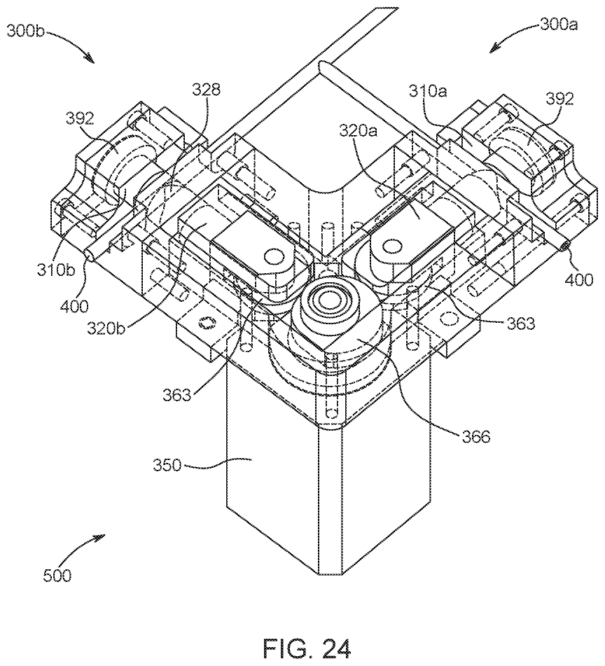

Referring now to FIG. 24, a valve assembly 500 is shown in accordance with another embodiment of the present disclosure. The valve assembly 500 of FIG. 24 may be substantially the same as the valve assembly 500 of FIGS. 14-18, except that a single cam 366 replaces the pair of cams 360a, 360b used in the valve assembly of FIGS. 14-18. The single cam 366 of the valve assembly 500 of FIG. 24 engages the anvils 320a, 320b, more particularly the cam follower bearings 363 of the anvils 320a, 320b, associated with both valves 300a, 300b and the cam 366 has a profile configured to selectively move the valves 300a, 300b between the open and closed positions. Rotation of the cam 366 thus controls selective opening and closing of both valves 300a, 300b. The components of the valve assembly 500 of FIG. 24 not specifically discussed herein are substantially identical to the corresponding components discussed in connection with the valve assembly 500 of FIGS. 14-18.

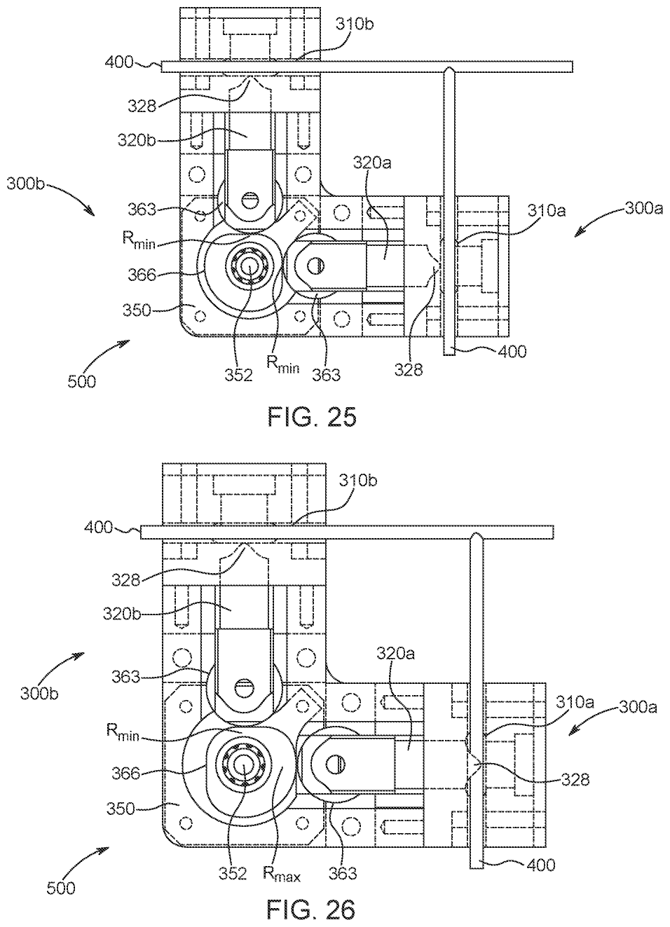

Referring now to FIGS. 25-28, the valve assembly 500 of FIG. 24 is shown in four positions corresponding to various states of the valves 300a, 300b. The various states of the valves 300a, 300b shown in FIGS. 25-28 correspond to various rotational positions of the motor shaft 352 and the cam 366 coupled thereto. Referring first to FIG. 25, the motor shaft 352 and the cam 366 are rotated such that both valves 300a, 300b are open. That is, the motor shaft 352 and the cam 366 are rotated such that a portion of minimum radius Rₘᵢₙ of the cam 366 engages the anvils 320a, 320b associated with both valves 300a, 300b. Both anvils are thus retracted away from the associated flexible tubing 400 and therefore the flexible tubing 400 associated with each valve 300a, 300b is substantially uncompressed and in the open position. Thus, fluid may flow through the flexible tubing 400 associated with each of the valves 300a, 300b. This position of valve assembly 500 may be used, for example, during a priming/purging operation to ensure that air is removed from the tubing 400 associated with each of valves 300a, 300b.

Referring next to FIG. 26, the motor shaft 352 and the cam 366 are rotated such that the first valve 300a is in the closed position and the second valve 300b is in the open position. That is, the motor shaft 352 and the cam 366 are rotated such that the portion of maximum radius Rₘₐₓ of the cam 366 engages the anvil 320a associated with the valve 300a, whereas the portion of minimum radius Rₘᵢₙ of the cam 366 engages the anvil 320b associated with the valve 300b. The anvil 320a engaging the portion of maximum radius Rₘₐₓ of the cam 366 is extended toward and compresses the flexible tubing 400 associated with the first valve 300a. The anvil 320b engaging the portion of minimum radius Rₘᵢₙ of the cam 366 is retracted away from the associated flexible tubing 400, and therefore the flexible tubing 400 associated with the second valve 300b is substantially uncompressed. Thus, fluid may flow through the flexible tubing 400 associated with the second valve 300b, but fluid may not flow through the flexible tubing 400 associated with the first valve 300a. The controller 200 (see FIG. 3) may be configured to move the valve assembly 500 to the position shown in FIG. 26 during an injection procedure. As shown in FIG. 3, the first valve 300a may be associated with the bulk fluid reservoir 120 and may be closed to prevent fluid from being injected into the fluid reservoir 120. The second valve 300b may be open to allow fluid to flow to the administration tube 176 and ultimately to the patient.

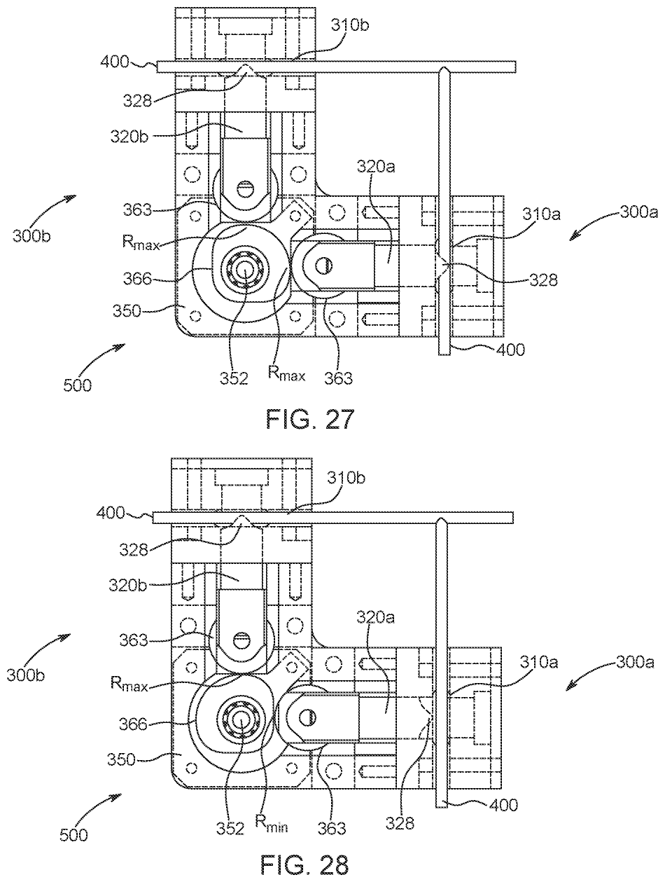

Referring next to FIG. 27, the motor shaft 352 and the cam 366 are rotated such that both valves 300a, 300b are in the closed position. That is, the motor shaft 352 and the cam 366 are rotated such that the portion of maximum radius Rₘₐₓ of the cam 366 engages the anvils 320a, 320b associated with both valves 300a, 300b. Engagement with the portion of maximum radius Rₘₐₓ of the cam 366 causes both anvils 320a, 320b to extend toward and compress the associated flexible tubing 400 and place both valves 300a, 300b in the closed position. Thus, fluid may not flow through the flexible tubing 400 associated with either of the valves 300a, 300b. The controller 200 (see FIG. 3) may be configured to move the valve assembly 500 to the position shown in FIG. 27 to stop all fluid flow downstream of the valve assembly 500. For example, the controller 200 may move the valve assembly 500 to the position shown in FIG. 27 in response to the at least one air detector 210 (see FIG. 3) detecting one or more air bubbles in the syringe 12 or the portion of tubing associated with the at least one air detector 210. Closure of the valves 300a, 300b as shown in FIG. 27 may prevent the one or more air bubbles detected by the at least one air detector 210 from being injected into the patient, may allow pre-pressurization of the syringe 12 before initiation of an injection procedure (i.e., pressurized fluid in the pressurized syringe cannot flow to either the patient or to the bulk fluid reservoir 120), and/or may be used to prevent fluid backflow from a pressurized syringe into a lower pressure syringe (e.g., a syringe 12 associated with the valve 500 shown in FIG. 27) during injection of fluid from the pressurized syringe into the patient.

Referring next to FIG. 28, the motor shaft 352 and the cam 366 are rotated such that a first valve 300a is in the open position and a second valve 300b is in the closed position. That is, the motor shaft 352 and the cam 366 are rotated such that the portion of minimum radius Rₘᵢₙ of the cam 366 engages the anvil 320a associated with the valve 300a, whereas a portion of maximum radius Rₘₐₓ of the cam 366 engages the anvil 320b associated with the valve 300b. The anvil 320a engaging the portion of minimum radius Rₘᵢₙ of the cam 366 is retracted away from the associated flexible tubing 400, and therefore the flexible tubing 400 associated with the first valve 300a is substantially uncompressed. The anvil 320b engaging the portion of maximum radius Rₘₐₓ of the cam 366 is extended toward and compresses the flexible tubing 400 associated with the second valve 300b. Thus, fluid may flow through the flexible tubing 400 associated with the first valve 300a, but fluid may not flow through the flexible tubing 400 associated with the second valve 300b. The controller 200 (see FIG. 3) may be configured to move the valve assembly 500 to the position shown in FIG. 28, for example, during a fill operation to allow fluid communication between the syringe 12 and a bulk fluid reservoir 120 while preventing fluid communication to the downstream tubing to the patient. As shown in FIG. 3, the first valve 300a may be associated with the bulk fluid reservoir 120 and may be open to allow fluid from the bulk fluid reservoir 120 to be drawn into the syringe 12. The second valve 300b may be closed to prevent fluid and/or air downstream of the valve 300b from being drawn into the syringe 12 during the fill operation.

Because the single cam 366 drives the anvils 320a, 320b associated with both valves 300a, 300b, the orientation of the anvils 320a, 320b must be selected such that the profile of the single cam 366 can effectively achieve the desired relative positions of the valves 300a, 300b. For example, in one embodiment, the anvils 320a, 320b of the pinch valves 300a, 300b may be oriented at approximately 90° relative to one another, as shown in FIGS. 24-28, to achieve the various valve positions shown in FIGS. 25-28. Other orientations of valves 300a, 300b may be used with suitable adjustment of the positions of the Rₘₐₓ, Rₘᵢₙ on cam 366.

In some embodiments, the cam 366 may have a profile such that rotation of the motor shaft 352 by approximately 90° moves the valve assembly 500 from the position shown in FIG. 25 (i.e., valve 300a open position, valve 300b open position) to the position shown in FIG. 26 (i.e., valve 300a closed position, valve 300b open position); a further rotation of the motor shaft 352 by approximately 90° moves the valve assembly 500 from the position shown in FIG. 26 to the position shown in FIG. 27 (i.e., valve 300a closed position, valve 300b closed position); a further rotation of the motor shaft 352 by approximately 90° moves the valve assembly 500 from the position shown in FIG. 27 to the position shown in FIG. 28 (i.e., valve 300a open position, valve 300b closed position); and a further rotation of the motor shaft 352 by approximately 90° moves the valve assembly 500 from the position shown in FIG. 28 to the position shown in FIG. 25. The motor shaft 352 may also be rotated in the reverse order, i.e. from the position shown in FIG. 25 to the position shown in FIG. 28; from the position shown in FIG. 28 to the position shown in FIG. 27; from the position shown in FIG. 27 to the position shown in FIG. 26; and from the position shown in FIG. 26 to the position shown in FIG. 25. Alternatively, the motor shaft 352 may also be rotated in any desired direction to reach the desired valve configuration required for a specific portion of the injection procedure.

The profile of the cam 366 may be selected and/or designed to reduce the torque demand on the motor 350 during opening/closing of the valves 300a, 300b; to decrease the time required for moving the valves 300a, 300b between the open and closed positions; and/or to optimize various other performance characteristics of the valves 300a, 300b.

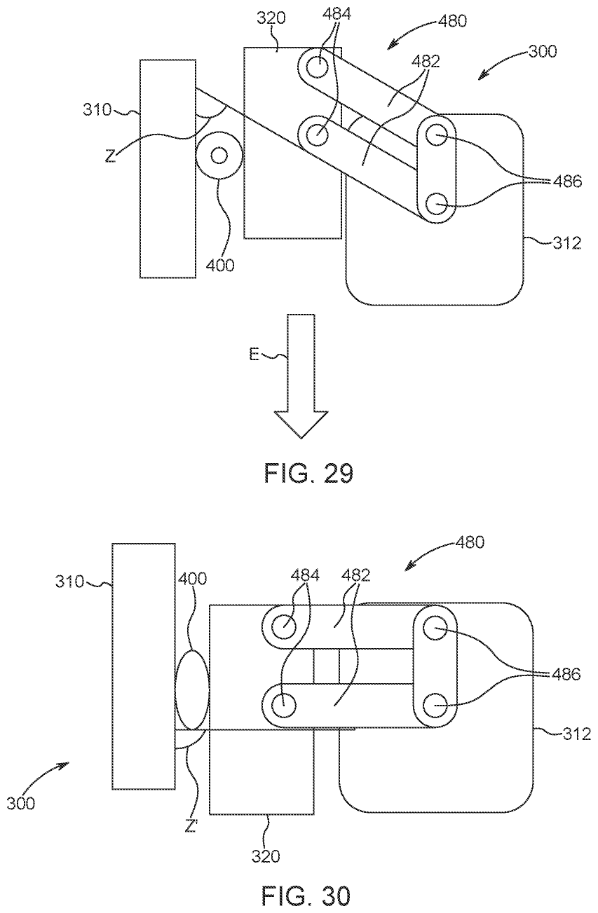

Referring now to FIGS. 29-30, a pinch valve 300 is shown in accordance with another embodiment of the present disclosure. In this embodiment of the valve 300, the anvil 320 is driven by a parallelogram linkage 480 to reversibly compress the associated flexible tubing 400. The parallelogram linkage 480 may include at least one pair of legs 482 that rotate relative to the frame 312 to drive the anvil 320 toward and away from the backing plate 310. Each of the legs 482 is rotatably connected to the anvil 320 at pivot points 484 and rotatably connected to the frame 312 at pivot points 486. In the open position of the valve 300 shown in FIG. 29, the parallelogram linkage 480 is retracted such that the legs 482 extend along an acute angle Z relative to the plane of the backing plate 310. In the open position, the flexible tubing 400 is substantially uncompressed by the anvil 320. To close the flexible tubing 400, the anvil 320 may be driven by a motor or actuator (not shown) in the direction of arrow E to the closed position shown in FIG. 30. When moving in the direction of arrow E, the anvil 320 may travel in a sweeping motion as the angle Z increases before ultimately reaching an angle Z′ (see FIG. 30). The anvil 320 is thus extended toward the backing plate 310 to compress the flexible tubing 400 between the anvil 320 and the backing plate 310. FIG. 30 shows the closed position of the valve 300 in which the anvil 320 is compressing the flexible tubing 400. In the embodiment shown in FIG. 30, the angle Z′ may be approximately 90°. In other embodiments, the angle Z′ may be less than 90° or more than 90°. In some embodiments, the anvil 320 may be driven until the controller 200 (see FIGS. 2-3) detects a predetermined resistance, e.g. a motor current, indicating that the flexible tubing 400 is compressed. As with other embodiments described herein, the backing plate 310 may include an associated biasing member (as described herein, not shown) that can control the force applied between the anvil 320 and the backing plate 310. The biasing force applied by the biasing element may be adjusted, as described herein, by a controller 200 according to features or properties associated with the injection protocol and/or the flexible tubing 400.

Due to friction between the anvil 320, the flexible tubing 400, and the backing plate 310, the flexible tubing 400 may be induced to roll and/or slip along the backing plate 310 as the flexible tubing 400 is engaged by the anvil 320 moving in the direction of arrow E. Rolling and/or slipping of the flexible tubing 400 may reduce stress on the flexible tubing 400 as the flexible tubing 400 is compressed. Further, rolling and/or slipping of the flexible tubing 400 may reduce the “dog-bone” effect. As described herein with reference to FIGS. 6-7, the term “dog-bone effect” is a phenomenon where the compressed cross-section of the lumen 404 of the flexible tubing 400 has a shape of a dog-bone (i.e., not fully compressed at the lateral sides of the lumen) due to increased material thickness at the lateral sides of the compressed flexible tubing 400 relative to the interior of the compressed flexible tubing 400.

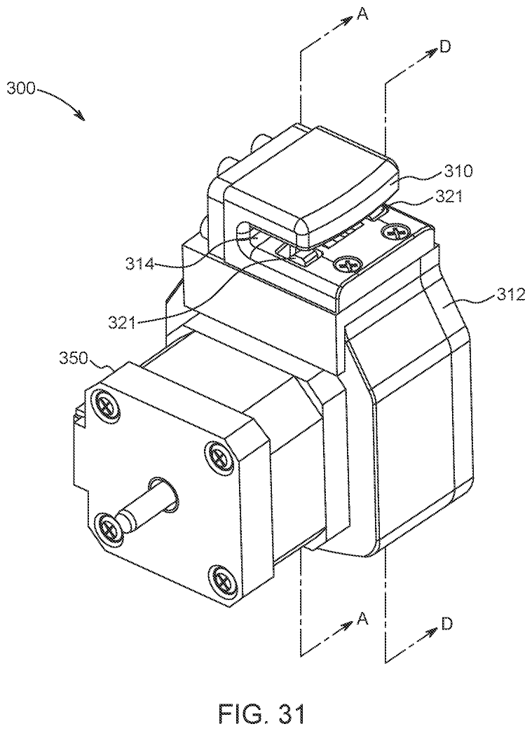

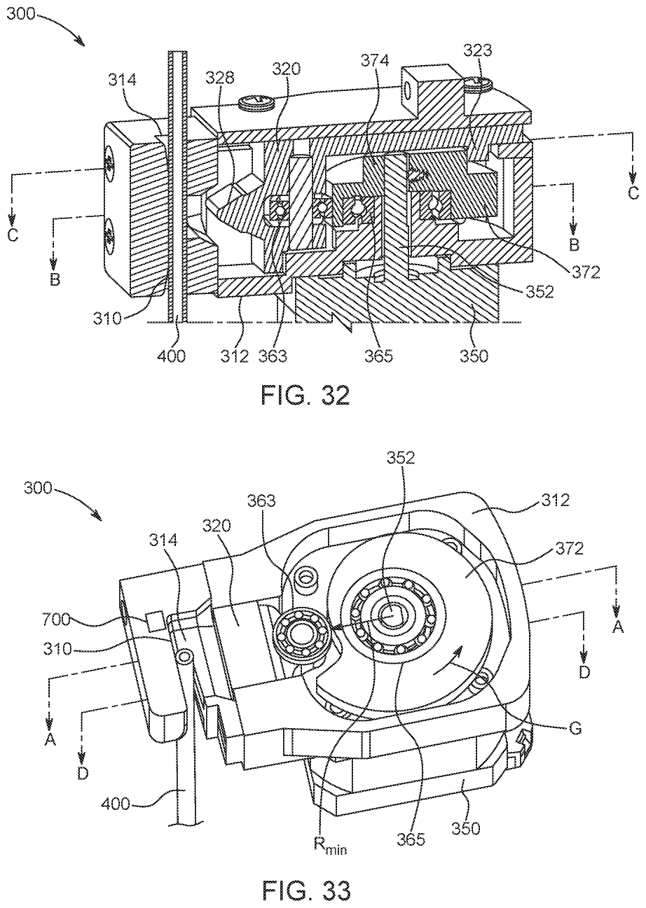

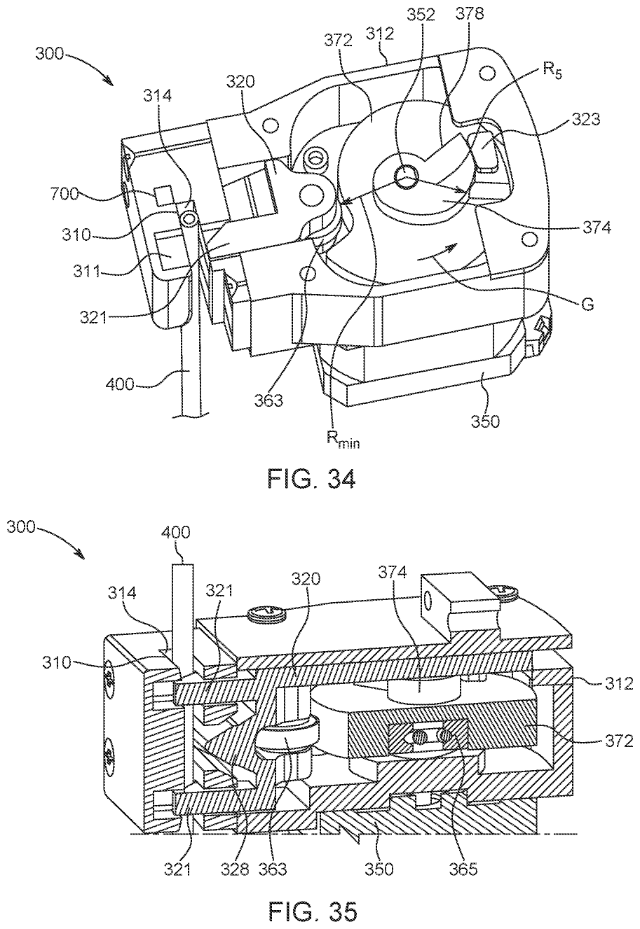

Referring now to FIGS. 31-41, a pinch valve 300 is shown in accordance with another embodiment of the present disclosure. In this embodiment, the valve 300 includes a two-part cam, including a primary cam 372 and a secondary cam 374, both coupled to the motor shaft 352. The primary cam 372 and the secondary cam 374 may be integrally formed or coupled to one another such that primary cam 372 and the secondary cam 374 rotate in unison with the motor shaft 352. A bearing 365 may be provided between the primary cam 372 and the frame 312 to provide support for the primary cam 372 and remove some or all of the radial load from the motor shaft 352. The primary cam 372 is configured to engage the anvil 320 to drive the anvil 320 toward the backing plate 310 to reversibly compress the associated flexible tubing 400. In the illustrated embodiment, the anvil 320 includes a cam follower bearing 363 configured to roll along the surface of the primary cam 372 as the primary cam 372 engages the anvil 320 to prevent wear on the anvil 320 and the primary cam 372. In a similar manner to the embodiments shown in FIGS. 14-27, the primary cam 372 has an irregular radius such that rotation of the primary cam 372 drives the anvil 320 toward the backing plate 310 to reversibly compress the flexible tubing 400, as will be described in greater detail with reference to FIGS. 34-41 herein.

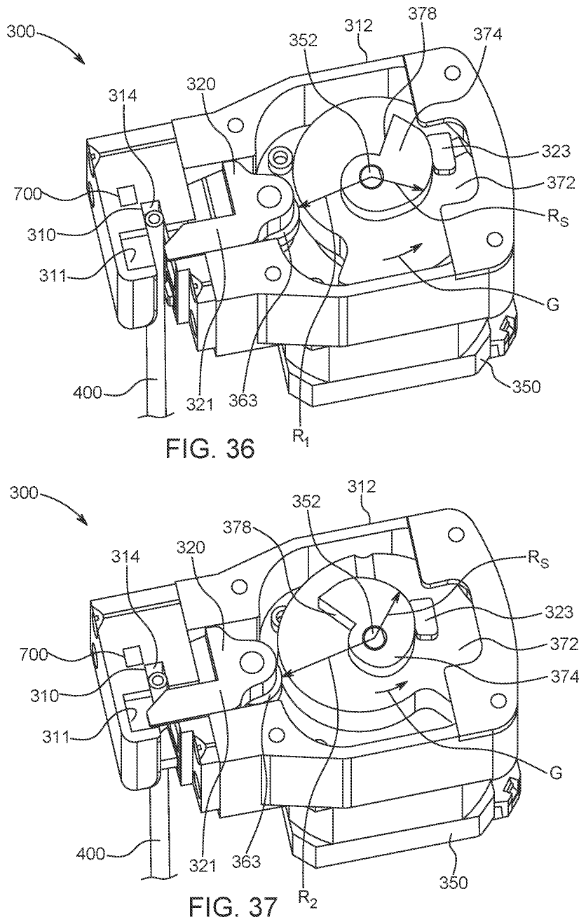

Referring in particular to FIG. 34, the valve 300 is shown in an open position in which a portion of minimum radius Rₘᵢₙ of the primary cam 372 engages the cam follower bearing 363. With the portion of minimum radius Rₘᵢₙ of the primary cam 372 engaging the cam follower bearing 363, the flexible tubing 400 is substantially or fully uncompressed. The radius of the primary cam 372 generally increases in a direction G in which the primary cam 372 is rotated such that rotation of the primary cam 372 drives the anvil 320 toward the backing plate 310. Referring now to FIG. 36, the primary cam 372 is shown in a first rotational position in which the primary cam 372 has been rotated in the direction G relative to the position shown in FIG. 34. At the position shown in FIG. 36, a radius R1 of the primary cam 372 engages the cam follower bearing 363. The radius R1 is greater than the portion of minimum radius Rₘᵢₙ, such that the primary cam 372 drives the anvil 320 toward the backing plate 310 and partially compresses the flexible tubing 400.

Referring now to FIG. 37, the primary cam 372 is shown in a second rotational position in which the primary cam 372 has been rotated in the direction G relative to the position shown in FIG. 36. At the position shown in FIG. 37, a radius R2 of the primary cam 372 engages the cam follower bearing 363. The radius R2 is greater than the radius R1, such that the primary cam 372 drives the anvil 320 further toward the backing plate 310 and further compresses the flexible tubing 400.

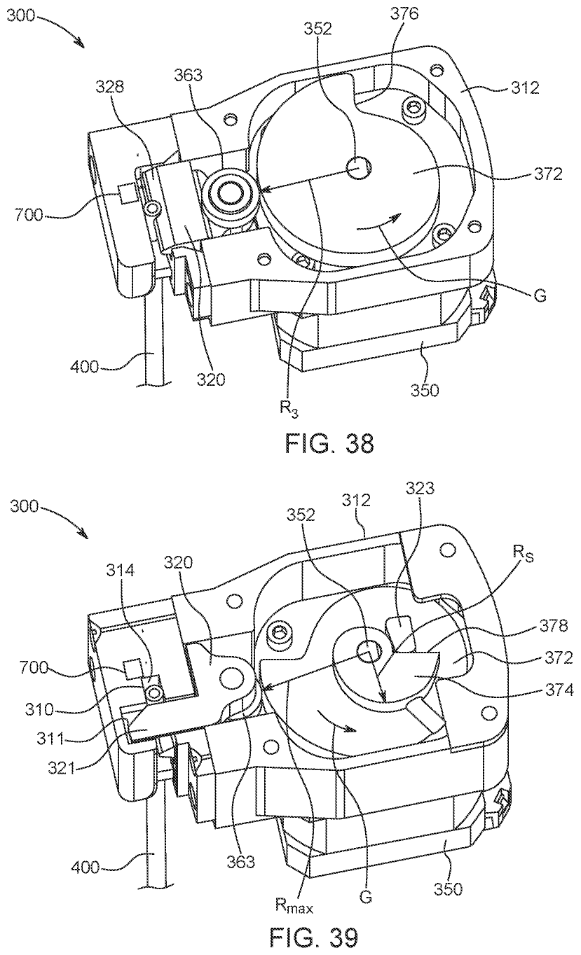

Referring now to FIG. 38, the primary cam 372 is shown in a third rotational position in which the primary cam 372 has been rotated in the direction G relative to the position shown in FIG. 37. At the position shown in FIG. 38, a radius R3 of the primary cam 372 engages the cam follower bearing 363. The radius R3 is greater than the radius R2, such that the primary cam 372 drives the anvil 320 further toward the backing plate 310 and further compresses the flexible tubing 400. Note that the section view shown in FIG. 38 omits the secondary cam 374, for clarity, so that the tip 328 of the anvil 320 can be seen engaging the flexible tubing 400.

Referring now to FIG. 39, the primary cam 372 is shown in a fourth rotational position, or a fully closed position, in which the primary cam 372 has been rotated in the direction G relative to the position shown in FIG. 38. At the position shown in FIG. 39, a portion of maximum radius Rₘₐₓ of the primary cam 372 engages the cam follower bearing 363, such that the primary cam 372 drives the anvil 320 further toward the backing plate 310 and fully compresses the flexible tubing 400. During rotation of the motor shaft 352, the primary cam 372 moves continuously from the first rotational position (shown in FIG. 34) to the fourth rotational position (shown in FIG. 39) through a plurality of intermediate positions having different radii between the Rₘᵢₙ and Rₘₐₓ.

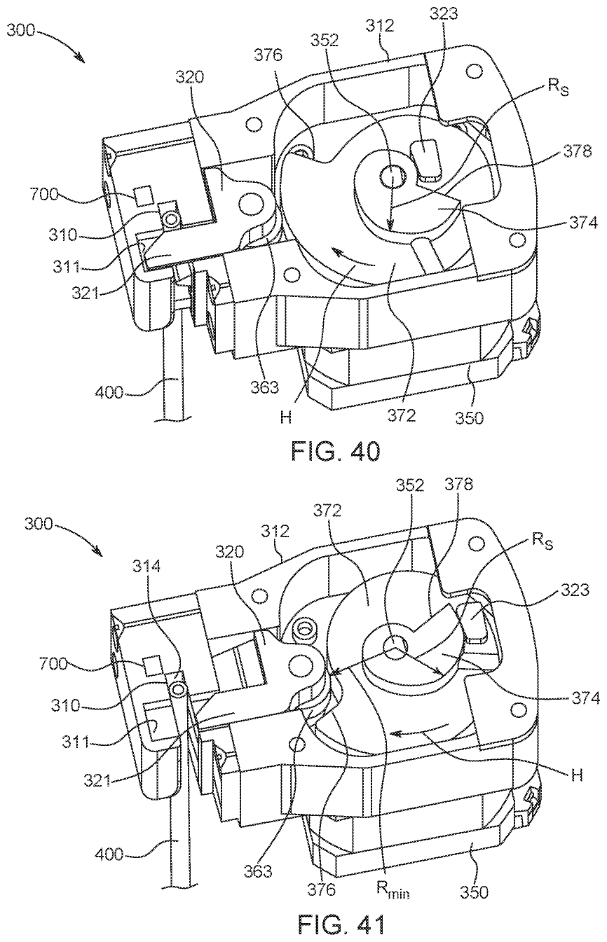

Referring now to FIG. 40, the primary cam 372 is shown in a fifth rotational position in which the primary cam is rotated in the direction H relative to the position shown FIG. 39 to allow the anvil 320 to move away from the backing plate 310 and partially decompress the flexible tubing 400. The direction H is rotationally opposite the direction G, and as such decompressing the flexible tubing 400 is substantially the opposite procedure to compressing the flexible tubing 400. Referring now to FIG. 41, the primary cam 372 is shown in a sixth rotational position, in which the primary cam is rotated in the direction H back to the open position shown in FIG. 34. The primary cam 372 includes a flat 376 which engages the cam follower bearing 363 to prevent further rotation of the primary cam 372 in direction H.

With continued reference to FIGS. 34-41, the secondary cam 374 rotates in unison with the primary cam 372 and engages a boss 323 of the anvil 320. The secondary cam 374 has an irregular radius Rs similar to the primary cam 372, however the radius Rs of the secondary cam 374 increases in the opposite rotational direction relative to the primary cam 372. That is, while the radius of the primary cam 372 increases in the rotational direction G, the radius of the secondary cam 374 increases in the opposite rotational direction H. The radius Rs of the secondary cam 374 is configured such that as the primary cam 372 is rotated to move the anvil 320, the boss 323 of the anvil 320 follows the radius Rs of the secondary cam 374, as illustrated in FIGS. 34-41. The secondary cam 374 is configured to drive the anvil 320 in the retraction direction, i.e. the direction away from the backing plate 310 to open the valve 300 and uncompress the flexible tubing 400. Referring again to FIG. 34, in the open position of the valve 300, the secondary cam 374 is oriented such that a portion of the secondary cam 374, where the radius Rs is at a maximum, faces and/or engages the boss 323 of the anvil 320. As the secondary cam 374 rotates along with the primary cam 372 in the direction G to close the valve 300, the radius Rs of the portion of the secondary cam 374 facing and/or engaging the boss 323 gradually decreases until a portion of the secondary cam 374 where the radius Rs is at a minimum faces and/or engages the boss 323 (as shown in FIG. 39). In some embodiments, there may be a small amount of clearance between the secondary cam 374 and the boss 323 throughout rotation of the secondary cam 374 in the direction G to prevent binding of the secondary cam 374 and the boss 323. Because the anvil 320 is driven toward the closed position (FIG. 39) by the primary cam 372 as the primary and secondary cams 372, 374 are rotated in the direction G, the secondary cam 374 need not directly engage the boss 323 during rotation in the direction G. Rather, when rotated in the direction G, the decreasing radius Rs of the secondary cam 374 allows the boss 323 to move in the direction toward the backing plate 310 with the rest of the anvil 310 under the influence of the primary cam 372.

The secondary cam 374 is configured to drive the anvil 320 to open the valve 300 when the primary and secondary cams 372, 374 are rotated in the direction H. Referring again to FIG. 39, when the valve 300 is in the closed position and fully compressing the flexible tubing 400, a portion of the secondary cam 374 where the radius Rs is at a minimum faces and/or engages the boss 323. Referring again to FIGS. 40-41, as the primary and secondary cams 372, 374 are rotated in the direction H, the primary cam 374 engages the boss 323 to drive the anvil 320 away from the backing plate 310 to the open position of the valve 300 (FIG. 41). In particular, as shown in the fifth rotational position of FIG. 40 a portion of the secondary cam 374 where the radius Rs is between a minimum and maximum value engages the boss 323, thereby driving the anvil 320 away from the backing plate 310. As the secondary cam 371 is rotated further in the direction H to the sixth rotational position, or the open position, of FIG. 41, a portion of the secondary cam 374 where the radius Rs is at a maximum engages the boss 323, thereby driving the anvil 320 to a farthest position from the backing plate 310. Between the positions shown in FIGS. 39-41, the radius Rs of the portion of the secondary cam 374 engaging the boss 323 continually increases as the secondary cam 374 is rotated in the direction H to continuous drive the anvil 320 away from the backing plate 310.

The secondary cam 374 may include a flat 378 that engages the boss 323 when the valve 300 is in the closed position shown in FIG. 39, such that the secondary cam 374, along with the primary cam 372 coupled thereto, cannot rotate further in the direction G.

With continued reference to FIGS. 31-41, the anvil 320 may include one or more fingers 321 having angled surfaces configured to engage the flexible tubing 400 and drive the tubing 400 into the slot 314 of the backing plate 310 as the flexible tubing 400 is compressed by the tip 328 of the anvil 320. The one or more fingers 321 extend from the anvil 320 such that the finger 321 move in the same direction as the anvil 320 (i.e., toward the backing plate 310) as the primary cam 372 is rotated in the direction G. As the anvil 320 is moved toward the backing plate 310, the fingers 321 extend between the flexible tubing 400 and the opening of the slot 314, as shown in FIG. 39, to prevent the flexible tubing 400 from moving out of the slot 314 in a direction perpendicular to the movement of the anvil 320. In addition, the fingers 321 may include angles surfaces which force the flexible tubing 400 further into the slot 314 in a direction perpendicular to the movement of the anvil 320. The backing plate 310 may include slots 311 for receiving the fingers 321 of the anvil 320 as the anvil 320 is driven towards the backing plate 310. The fingers 321 retain the flexible tubing 400 against the backing plate 310 and prevent flexible tubing 400 from being inadvertently removed from backing plate 310.

Referring again to FIGS. 31-41, the valve 300 may include a tubing detector 700 configured to detect the presence or absence of the flexible tubing 400 on the backing plate 310. The tubing detector 700 may include a proximity sensor, optical sensor, pressure sensor, pressure plate, limit switch, etc. to detect the presence or absence of the flexible tubing 400 when the tubing is inserted into or present in the slot 314. The tubing detector 700 may be in operative communication with the controller 200 (see FIGS. 2 and 3). The controller 200 may be programmed or configured to prevent rotation, halt, or abort an injection procedure in response to determining that the flexible tubing 400 is not present on the backing plate 310.

Components of the embodiment illustrated in FIGS. 31-41 but not specifically described herein are understood to be similar to like features of the other embodiments described herein.

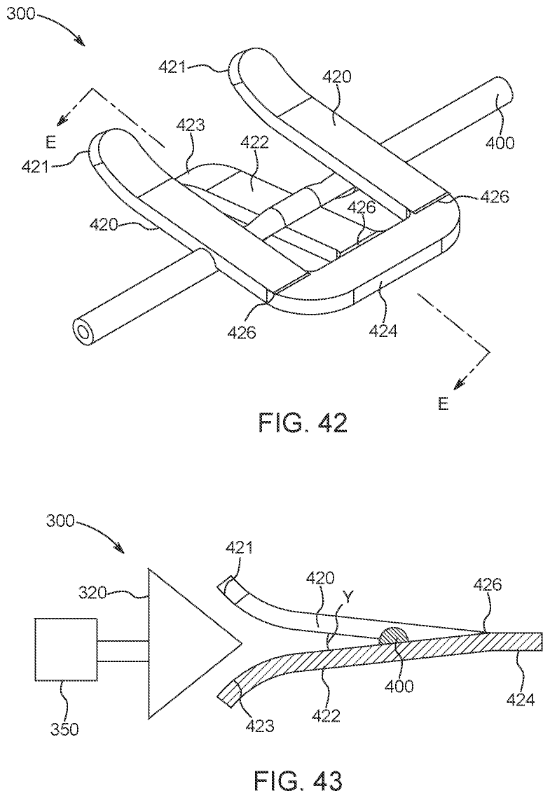

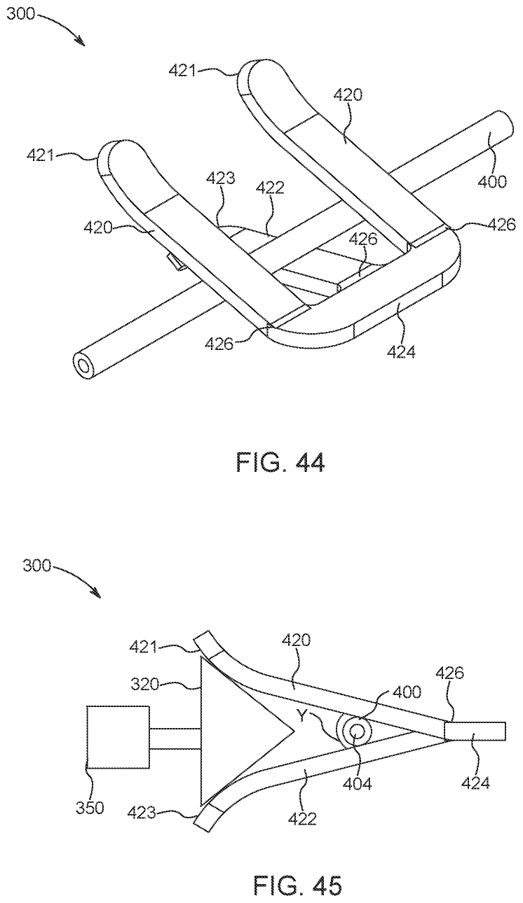

Referring now to FIGS. 42-45, a pinch valve 300 is shown in accordance with another embodiment of the present disclosure. In this embodiment, the valve 300 includes at least one upper flexible arm 420 and at least one lower flexible arm 422. The at least one upper flexible arm 420 and the at least one lower flexible arm 422 may be connected to a baseplate 424 such that the associated flexible tubing 400 may be arranged between the at least one upper flexible arm 420 and the at least one lower flexible arm 422. The connection between the baseplate 424 and the flexible arms 420, 422 may include a hinge portion 426, such as a living hinge, that allows the upper and lower flexible arms 420, 422 to be deflected outwardly away from one another. The at least one upper flexible arm 420 and the at least one lower flexible arm 422 may be biased towards one another such that the flexible tubing 400 is reversibly compressed between the flexible arms 420, 422 in a relaxed position of the valve 300 as shown in FIGS. 42-43. To open the flexible tubing 400, an anvil 320 may be driven between the at least one upper flexible arm 420 and the at least one lower flexible arm 422 to relieve the pressure applied by the flexible arms 420, 422 against the flexible tubing 400. The anvil 320 may engage the camming surfaces 421, 423 of the flexible arms 420, 422 to spread the at least one upper flexible arm 420 and the at least one lower flexible arm 422 apart from one another, such that an angle Y between the flexible arms 420, 422 increases as the anvil 320 is driven toward the baseplate 426 as shown in FIGS. 44-45. The anvil 320 may be driven by a motor 350 such as a linear actuator, solenoid, cam, or other conventional electromechanical motor in operative communication with the controller 200 (see FIGS. 2 and 3). Alternatively, the at least one upper flexible arm 420 and the at least one lower flexible arm 422 may be deflected away from each other manually, for example by a user applying a force to move the at least one upper flexible arm 420 and the at least one lower flexible arm 422 away from each other to an open position shown in FIG. 44.

Each of the various embodiments of the valves 300, 300a, 300b described herein may be configured for rapid response. That is, in response to detection of one or more air bubbles in the fluid line by the at least one air detector 210 (see FIGS. 2-3), the controller 200 of the fluid injection system 1000 may rapidly move the valves 300, 300a, 300b from the open position to the closed position to prevent the air bubble from flowing into the vasculature of a patient. For example, in certain procedures such as angiography injection procedures pressures as high as 1200 psi may be utilized. At injection pressures of 1200 psi and fluid flow rates of at 25 to 40 mL/sec, an air bubble can travel up to 4 feet in a tube path depending on the inner diameter (ID) of the tubing. For example, at approximately 1200 psi, an air bubble may travel a distance corresponding to 3.2 mL over 80 milliseconds at a flow rate of 30 mL/sec in a tubing with a 0.072 inch ID. The distance equivalence of 3.2 mL volume for such an embodiment may be approximately 4 feet of tubing length travelled during 80 milliseconds. In various embodiments, the valves 300, 300a, 300b according to the various embodiments may be moved from the open position to the closed position in less than about 200 milliseconds, for example from about 40 milliseconds to about 100 milliseconds or in other embodiments from about 40 milliseconds to about 80 milliseconds. This rapid response time may ensure the valves 300, 300a, 300b can attain the closed position upon receiving a signal from the controller 200 prior to air reaching the patient, particularly for high pressure injection procedures.

In any of the various embodiments of the valves 300, 300a, 300b described herein, the anvil 320 and/or backing plate 310 may include surface features to improve engagement with the flexible tubing 400. For example, the surface of the anvil 320 and/or the backing plate 310 may have a textured surface to increase the grip of the anvil 320 and/or backing plate 310 against the flexible tubing 400. According to certain embodiments, the textured surface may have a surface roughness in a range of between 2 microinches and 125 microinches, or in other embodiments, in a range between 20 microinches and 75 microinches. In some embodiments, the textured surface of the anvil 320 and/or the backing plate 310 may be configured to assist in maintaining a position of flexible tubing 400 relative to backing plate 310 and/or anvil 320.

The various embodiments of the valves 300, 300a, 300b described herein may be configured and used as a shut-off valve, a flow rate control valve, or a combination thereof. When configured as a shut-off valve, the anvil 320 of the pinch valve 300, 300a, 300b may be moved between the open position (shown, e.g., in FIG. 4), in which the flexible tubing 400 is substantially uncompressed, and the closed position (shown, e.g., in FIG. 5), in which the flexible tubing 400 is fully compressed.

When configured as a flow rate control valve, the valve 300, 300a, 300b described herein may be moved between a plurality of partially open and/or partially closed positions to reversibly compress the flexible tubing by varying degrees. For example, the valves 300, 300a, 300b described herein may be moved to any of a finite or infinite number of positions between the open position (shown, e.g., in FIG. 4) and the fully closed position (shown, e.g., in FIG. 5) and thus, provide control of the fluid flow through the reduced cross-section of the lumen 404. The cross-sectional area of the lumen 404 of the flexible tubing 400 may therefore be controlled depending on the position of the valve 300, 300a, 300b, specifically the position of the anvil 320 relative to the backing plate 310. According to certain embodiments, the cross-sectional area of the lumen 404 of the flexible tubing 400 may be chosen to correspond to a known and/or empirically-derived flow rate of fluid through the flexible tubing 400. In some embodiments, the valve 300, 300a, 300b may include an encoder to determine the position of the anvil 320 and/or the backing plate 310, and the controller 200 may be configured to actuate the valve motor 350 to move the anvil or backing plate 310 to a desired position associated with a desired flow rate of fluid through the flexible tubing 400. In embodiments, the fluid injector system 1000 may include at least one flow rate detector 600 (see FIG. 2) downstream of the valve 300, 300a, 300b to measure the flow of fluid through the valve 300, 300a, 300b. The controller 200 may actuate the motor 350 to move the anvil 320 or the backing plate 310 of the valve 300, 300a, 300b to achieve a desired flow rate as measured by the at least one flow rate detector 600. In some embodiments, the controller may be configured to adjust the position of the valve 300, 300a, 300b in real time to control the flow rate based on a signal received from the at least one flow rate detector 600.

In some embodiments, the valve 300, 300a, 300b may be controlled by the controller 200 based on motor current. In particular, the controller 200 may be configured to move the anvil 320 or backing plate 310 until a predetermined current is drawn by the motor 350, corresponding to the open position, the closed position, or any other at least partially closed position, of the valve 300, 300a, 300b. Measurement of the motor current may take into account creep experienced over the useable life of the flexible tubing 400, which may change the force required to compress the flexible tubing 400. For example, in certain embodiments, the controller 200 may utilize the flow values measured at the at least one downstream flow rate detector 600 to benchmark the motor current and position of valves 300, 300a, 300b with a flow rate of fluid in the downstream fluid path and update amount of movement of anvil 320 necessary to achieve a desired flow rate or to fully compress or fully open flexible tubing 400.

While examples of fluid injector systems, valves and valve assemblies, and methods of operation thereof were provided in the foregoing description, those skilled in the art may make modifications and alterations to these examples without departing from the scope and spirit of the disclosure. Accordingly, the foregoing description is intended to be illustrative rather than restrictive. The disclosure described hereinabove is defined by the appended claims, and all changes to the disclosure that fall within the meaning and the range of equivalency of the claims are to be embraced within their scope.

1. A fluid injector system, comprising:
at least one injector for pressurizing and delivering at least one fluid from at least one fluid reservoir;
a first flexible tube having a first lumen, the first flexible tube in fluid communication with the at least one fluid reservoir and configured for fluid communication with a bulk fluid source;
a second flexible tube having a second lumen, the second flexible tube in fluid communication with the at least one fluid reservoir and configured for fluid communication with a patient administration line; and
a valve assembly configured to selectively and reversibly compress the first flexible tube and the second flexible tube to open and close the first lumen and the second lumen, the valve assembly comprising:
a first anvil moveable between a retracted position in which the first lumen is at least partially open and an extended position in which the first anvil reversibly compresses the first flexible tube to close the first lumen;
a second anvil moveable between a retracted position in which the second lumen is at least partially open and an extended position in which the second anvil reversibly compresses the second flexible tube to close the second lumen; and
at least one eccentric cam rotatable to move the first anvil and the second anvil between the retracted position and the extended position.

2. The fluid injector system of claim 1, wherein the valve assembly is movable between:
a first position in which the first lumen and the second lumen are at least partially open;
a second position in which the valve assembly closes the first lumen and the second lumen is at least partially open;
a third position in which the first lumen is at least partially open and the valve assembly closes the second lumen; and
a fourth position in which the valve assembly closes the first lumen and the second lumen.

3. The fluid injector system of claim 2, wherein the first anvil is moveable along a first axis, and wherein the second anvil is moveable along a second axis.

4. The fluid injector system of claim 3, wherein the first axis is oriented at approximately 90° relative to the second axis.

5. The fluid injector system of claim 1, wherein the valve assembly further comprises:
a first biasing element biasing the first anvil toward the extended position; and
a second biasing element biasing the second anvil toward the extended position.

6. The fluid injector system of claim 1, wherein the valve assembly further comprises:
a first backing plate biased toward the first flexible tube to provide a pressure against the first flexible tube; and
a second backing plate biased toward the second flexible tube to provide a pressure against the second flexible tube.

7. The fluid injector system of claim 6, where the pressures provided by the first backing plate and the second backing plate are adjustable.

8. The fluid injector system of claim 1, wherein the at least one eccentric cam comprises a single cam engaging both the first anvil and the second anvil.

9. The fluid injector system of claim 1, wherein the at least one eccentric cam comprises:
a first cam engaging the first anvil; and
a second cam engaging the second anvil,
wherein the first cam and the second cam share a rotation axis.

10. The fluid injector system of claim 1, wherein the at least one eccentric cam comprises at least one constant radius section,
wherein, with the first anvil engaging the constant radius section, rotation of the at least one eccentric cam over a span of the constant radius section does not move the first anvil between the retracted position and the extended position, and
wherein, with the second anvil engaging the constant radius section, rotation of the at least one eccentric cam over a span of the constant radius section does not move the second anvil between the retracted position and the extended position.

11. (canceled)

12. The fluid injector system of claim 1, wherein the valve assembly further comprises a backing plate having a groove for receiving the first flexible tube, and wherein the first anvil comprises a projection for reversibly compressing the first flexible tube against the groove of the backing plate.

13. The fluid injector system of claim 1, further comprising:
at least one air detector associated with at least one of the first flexible tube and the second flexible tube; and
a controller programmed or configured to move the valve assembly to the fourth position in response to detection of at least one air bubble by the at least one air detector.

14. A fluid injector system, comprising:
at least one injector for pressurizing and delivering at least one fluid from at least one fluid reservoir;
a first flexible tube having a first lumen, the first flexible tube in fluid communication with the at least one fluid reservoir and configured for fluid communication with a bulk fluid source; and
a valve assembly configured to selectively and reversibly compress the first flexible tube to open and close the first lumen, the valve assembly comprising:
a first backing plate configured for receiving the first flexible tube;
a first anvil moveable between a retracted position and an extended position, wherein, in the extended position, the first anvil is configured to reversibly compress the first flexible tube against the first backing plate; and
at least one driving element configured to move the first anvil between the retracted position and the extended position,
wherein the first backing plate is biased toward the first flexible tube to provide a pressure against the first flexible tube.

15.-35. (canceled)

36. A valve for a fluid injector system, the valve comprising:
a backing plate configured for receiving a flexible tube;
an anvil moveable between a retracted position and an extended position, wherein, in the extended position, the anvil is configured to reversibly compress the flexible tube against the backing plate;
a primary cam configured to engage the anvil to move the anvil from the retracted position and the extended position; and
a secondary cam configured to engage a boss of the anvil to move the anvil from the extended position to the retracted position.

37. The valve of claim 36, wherein a radius of the primary cam increases in a first direction of rotation, and wherein a radius of the secondary cam increases in a second directing of rotation opposite the first direction of rotation.

38. The valve of claim 36, wherein the primary cam and the secondary cam are configured to rotate in unison.

39. The valve of claim 36, wherein at least one of a portion of the primary cam having a maximum radius engages the anvil when the anvil is in the extended position and a portion of the secondary cam having a maximum radius engages the boss when the anvil is in the retracted position.

40. The valve of claim 36, wherein at least one of a portion of the secondary cam having a minimum radius engages the boss when the anvil is in the extended position and a portion of the primary cam having a minimum radius engages the anvil when the anvil is in the retracted position.

41. (canceled)

42. (canceled)

43. The valve of claim 36, wherein the anvil comprises at least one finger configured to engage the flexible tube in the retracted position to retain the flexible tube against the backing plate.

44. The valve of claim 36, further comprising a tubing detector configured to detect the presence or absence of the flexible tube on the backing plate.

45. (canceled)
